SEQUENCE LISTING (98 sequences)

SEQ ID NO: 1 - protein, 12 aa, unidentified
MPLRYPTQVTLD

SEQ ID NO: 2 - protein, 12 aa, unidentified
DGSMLNRMRGFS

SEQ ID NO: 3 - protein, 12 aa, unidentified
APTTWFNSDSIT

SEQ ID NO: 4 - protein, 12 aa, unidentified
FKQDAWEAVDIR

SEQ ID NO: 5 - protein, 12 aa, unidentified
EPELDNARVLQI

SEQ ID NO: 6 - protein, 12 aa, unidentified
MADQTMGLHHGM

SEQ ID NO: 7 - protein, 12 aa, unidentified
AFSTTHRYSELI

SEQ ID NO: 8 - protein, 12 aa, unidentified
STMTESIRQRWD

SEQ ID NO: 9 - protein, 12 aa, unidentified
AISPSRYFYDET

SEQ ID NO: 10 - protein, 12 aa, unidentified
FKQDAWEAVDIR

SEQ ID NO: 11 - protein, 12 aa, unidentified
EPELDNARVLQI

SEQ ID NO: 12 - protein, 12 aa, unidentified
YALGRPSLQGPN

SEQ ID NO: 13 - protein, 12 aa, unidentified
DGSMLNRMRGFS

SEQ ID NO: 14 - protein, 12 aa, unidentified
DNHTTDLWNFSH

SEQ ID NO: 15 - protein, 12 aa, unidentified
QQMHEWYFQSLP

SEQ ID NO: 16 - protein, 12 aa, unidentified
MPLRYPTQVTLD

SEQ ID NO: 17 - protein, 12 aa, unidentified
STTAVSPVNSAK

SEQ ID NO: 18 - protein, 12 aa, unidentified
APTTWFNSDSIT

SEQ ID NO: 19 - protein, 12 aa, unidentified
QELHYGLSSKRF

SEQ ID NO: 20 - protein, 12 aa, unidentified
SVSVNNATLYFY

SEQ ID NO: 21 - protein, 12 aa, unidentified
HGDGIKMMRQRM

SEQ ID NO: 22 - protein, 12 aa, unidentified
SMPRLWDNTSNT

SEQ ID NO: 23 - protein, 12 aa, unidentified
SLELKRSDVQHA

SEQ ID NO: 24 - protein, 12 aa, unidentified
AMTDPWLSDSRS

SEQ ID NO: 25 - protein, 12 aa, unidentified
YSTVYPVGQFPQ

SEQ ID NO: 26 - protein, 12 aa, unidentified
TTLWLDRDEALK

SEQ ID NO: 27 - protein, 12 aa, unidentified
FSTDYAWTAEAT

SEQ ID NO: 28 - protein, 12 aa, unidentified
SANYNVQAGWTH

SEQ ID NO: 29 - protein, 12 aa, unidentified
SPKSMTTGTVVV

SEQ ID NO: 30 - protein, 12 aa, unidentified
MADQTMGLHHGM

SEQ ID NO: 31 - protein, 12 aa, unidentified
ASSPRHSFAMGS

SEQ ID NO: 32 - protein, 12 aa, unidentified
ISNAVNEIRTLR

SEQ ID NO: 33 - protein, 12 aa, unidentified
AHNEDKWVTHRS

SEQ ID NO: 34 - protein, 12 aa, unidentified
GTVSYMVKMDNN

SEQ ID NO: 35 - protein, 12 aa, unidentified
FNSDSRSTHQED

SEQ ID NO: 36 - protein, 12 aa, unidentified
NQCKECFIRAGD

SEQ ID NO: 37 - protein, 12 aa, unidentified
FGLEPRANLHFT

SEQ ID NO: 38 - protein, 12 aa, unidentified
KIYTITSSHDSY

SEQ ID NO: 39 - protein, 12 aa, unidentified
SNSHPNWVELRP

SEQ ID NO: 40 - protein, 12 aa, unidentified
WDSLSYPWRITL

SEQ ID NO: 41 - protein, 12 aa, unidentified
IATQKIHVDTKQ

SEQ ID NO: 42 - protein, 12 aa, unidentified
WDTSWNFPDQPD

SEQ ID NO: 43 - protein, 12 aa, unidentified
GGWQSLWDKPEH

SEQ ID NO: 44 - protein, 12 aa, unidentified
VAGNKSVSTSHI

SEQ ID NO: 45 - protein, 12 aa, unidentified
GHAHSLTQNPLF

SEQ ID NO: 46 - protein, 12 aa, unidentified
EQWYYSPPIYAN

SEQ ID NO: 47 - protein, 12 aa, unidentified
VDKQHHTAVFPH

SEQ ID NO: 48 - protein, 12 aa, unidentified
VDIKSHFSHTGK

SEQ ID NO: 49 - protein, 12 aa, unidentified
VVDPNHSVWILS

SEQ ID NO: 50 - protein, 12 aa, unidentified
FSDAGNLPANAM

SEQ ID NO: 51 - protein, 12 aa, unidentified
HTKLLTYTKSTG

SEQ ID NO: 52 - protein, 12 aa, unidentified
YPTTANRMYTSY

SEQ ID NO: 53 - protein, 12 aa, unidentified
IGKSSNAPSLRA

SEQ ID NO: 54 - protein, 12 aa, unidentified
DGQVSDHSYVQW

SEQ ID NO: 55 - protein, 12 aa, unidentified
DKYEFFSPTQHY

SEQ ID NO: 56 - protein, 12 aa, unidentified
SLQTLRLGHNTS

SEQ ID NO: 57 - protein, 12 aa, unidentified
WDMWPSMDWKAE

SEQ ID NO: 58 - protein, 12 aa, unidentified
AEPVRDRTQTPK

SEQ ID NO: 59 - protein, 12 aa, unidentified
EMHKRVLTWNMI

SEQ ID NO: 60 - protein, 12 aa, unidentified
SVFSSFAIFMDH

SEQ ID NO: 61 - protein, 12 aa, unidentified
QIEPGDLEHYPT

SEQ ID NO: 62 - protein, 12 aa, unidentified
VNRSGIETGAVK

SEQ ID NO: 63 - protein, 12 aa, unidentified
KVSMQADFWSVM

SEQ ID NO: 64 - protein, 12 aa, unidentified
EMHGMPLPMLLA

SEQ ID NO: 65 - protein, 12 aa, unidentified
NGAYSLAIRYTS

SEQ ID NO: 66 - protein, 12 aa, unidentified
WAMDGQQHSNNY

SEQ ID NO: 67 - protein, 12 aa, unidentified
GATSVGSVAMQN

SEQ ID NO: 68 - protein, 12 aa, unidentified
AYAAMGGPSQEK

SEQ ID NO: 69 - protein, 12 aa, unidentified
SLKPPMHYANMY

SEQ ID NO: 70 - protein, 12 aa, unidentified
SYDGTMLKQVRL

SEQ ID NO: 71 - protein, 12 aa, unidentified
LDMYSYIETYNS

SEQ ID NO: 72 - protein, 12 aa, unidentified
THDMGKMDRTTT

SEQ ID NO: 73 - protein, 12 aa, unidentified
ADNVLRRALENI

SEQ ID NO: 74 - protein, 12 aa, unidentified
SVPAKKAIDWYS

SEQ ID NO: 75 - protein, 12 aa, unidentified
VHASGFRDSQRW

SEQ ID NO: 76 - protein, 12 aa, unidentified
THKLSDWEDIGV

SEQ ID NO: 77 - protein, 12 aa, unidentified
GSHNTYIESTIP

SEQ ID NO: 78 - protein, 12 aa, unidentified
GFPVPTKEGNRH

SEQ ID NO: 79 - protein, 12 aa, unidentified
IPLENQHKIYST

SEQ ID NO: 80 - protein, 12 aa, unidentified
SKISNMQGLHRT

SEQ ID NO: 81 - protein, 12 aa, unidentified
KDGMPWNIGYSR

SEQ ID NO: 82 - protein, 12 aa, unidentified
QSRTYSGAELLT

SEQ ID NO: 83 - protein, 1190 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATIAISPSRYFYDETNSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 84 - protein, 1190 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSAPTTWFNSDSITRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 85 - protein, 1190 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGMPLRYPTQVTLDSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 86 - protein, 1190 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTSANYNVQAGWTHFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 87 - protein, 1190 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRDGSMLNRMRGFSRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 88 - protein, 1190 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGWAMDGQQHSNNYNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 89 - protein, 1190 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIEPELDNARVLQIQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 90 - protein, 1190 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTAPTTWFNSDSITSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 91 - protein, 1190 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRAPTTWFNSDSITELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 92 - protein, 1190 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDWAMDGQQHSNNYVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 93 - protein, 1202 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATIAPTTWFNSDSITNSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRAPTTWFNSDSITRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 94 - protein, 1214 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATIAPTTWFNSDSITNSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRAPTTWFNSDSITRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIAPTTWFNSDSITQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 95 - protein, 1226 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATIAPTTWFNSDSITNSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRAPTTWFNSDSITRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIAPTTWFNSDSITQNRGYIEVPIHFPSTAPTTWFNSDSITSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 96 - protein, 1202 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATIMPLRYPTQVTLDNSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYREPELDNARVLQIRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 97 - protein, 1214 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATIMPLRYPTQVTLDNSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYREPELDNARVLQIRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIMADQTMGLHHGMQNRGYIEVPIHFPSTSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

SEQ ID NO: 98 - protein, 1226 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATIMPLRYPTQVTLDNSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYREPELDNARVLQIRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIMADQTMGLHHGMQNRGYIEVPIHFPSTAPTTWFNSDSITSTRYRVRVRYASVTPIHLNVNWGNSSIFSNTVPATATSLDNLQSSDFGYFEGANAFTSSLGDIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCELNRGYRDYTPLPVGYVTKELEYFPETDKVWIEIGETEGTFIVDSVELLLMEE

(19) United States
(12) Patent Application Publication
(10) Pub. No.: US 2026/0020575 A1
(43) Pub. Date: Jan. 22, 2026

(54) MODIFIED INSECTICIDAL PROTEINS WITH IMPROVED TOXICITY AGAINST LEPIDOPTERAN INSECTS

(71) Applicant: Ajeet Seeds Pvt. Ltd., Aurangabad (IN)

(72) Inventors: Purushottam RAJABHAU LOMATE, Aurangabad (IN); Prashant SHIVASHARAN PYATI, Aurangabad (IN); Shuban KISHEN RAWAL, Aurangabad (IN)

(73) Assignee: Ajeet Seeds Pvt. Ltd., Aurangabad (IN)

(21) Appl. No.: 18/996,611

(22) PCT Filed: Sep. 6, 2023
(86) PCT No.: PCT/IN2023/050839

§ 371 Date: Jan. 17, 2025

(30) Foreign Application Priority Data
Sep. 8, 2022 (IN) 202221051330

(51) Int. Cl. A01N 63/50; A01P 7/04; C07K 14/325; C07K 14/435

(52) U.S. Cl. CPC A01N 63/50; A01P 7/04; C07K 14/325; C07K 14/43563; C07K 2319/00

(57) ABSTRACT

The present invention provides modified Cry1Ac protein having enhanced toxicity towards resistant pink bollworm (PBW) species. Insertion of said specific, tightly binding gut peptides in Cry1Ac at one or more identified sites increases the retention of toxin in the PBW gut. resulting in solubilisation and pore formation leading to sepsis and larval death. The modified Bt toxin exhibits significant toxicity against resistant pink bollworm and thus can be used for generation of insect resistant transgenic plants.

### FIELD OF INVENTION

The present invention relates to the field of molecular biology. Specifically, the present invention relates to development of modified insecticidal toxins with improved toxicity to control Lepidopteran insect pests.

### BACKGROUND OF THE INVENTION

Lepidopteran insects present one of the greatest challenges for insect pest management in Indian agriculture and cause severe damage to important crop plants. Bacillus thuringiensis (Bt) toxins are widely accepted insecticidal proteins and highly efficient/effective in controlling Lepidopteran insects especially on cotton. Bt crops have decreased reliance on conventional insecticides by, suppressing key insect pests, and increased yield adding to farmer's profit. However, over the period of time, evolution of resistance by insect pests has reduced the benefits of insecticidal proteins from Bt that are used extensively in sprays and transgenic crops. Pink bollworm, Pectinophora gossypiella (Lepidoptera) is a global pest of cotton and currently a major threat for cotton cultivation in India. Some of the recent reports (Tabashnik et al., 2013, Wang et al., 2022) have revealed that field evolved resistance to Bt cotton, Bollguard II, in pink bollworms, could be a major retaliation to Bt cotton technology, the only transgenic technology available against pink bollworm at present.

It is well known that proteolytic activation of Bt toxins, and the subsequent binding to the insect gut epithelium are two important steps involved in the mechanism of Bt Cry protein toxicity. However, mutations in a gene encoding cadherin protein, a receptor of Cry1Ac in pink bollworm gut has led to field-evolved resistance of pink bollworm to Cry1Ac toxins (Fabrick et. al., 2009). Hence, altered or no binding of toxin to the gut receptors is the principal reason behind field evolved resistance in pink bollworm.

To withstand field evolved resistance in pink bollworm, Bt cotton technology needs to be continuously upgraded by introduction of novel or improved Bt Cry toxins with enhanced insecticidal activity. As field evolved Bt resistance in pink bollworm is attributed to altered or no binding of toxin to the gut receptors, increasing binding of Cry toxins to insect gut receptors by various means would be the prudent approach ahead.

This problem can be addressed by modifying the binding target(s), specificity and/or affinity of Bt toxins with the ultimate goal of producing designer toxins that target resistant species and counter the field evolved resistance. The use of synthetic gut binding peptides (GBPs) in the engineering of Bt toxins is a relatively new approach, which has the potential to restore and/or enhance the toxicity of Bt toxins against pests which have evolved resistance to the Bt toxin (Deist et al., 2014).

Advances in powerful combinatorial technologies such as phage display library (PDL) suggest new approaches for selecting binding peptides (Deist et al., 2014 and Sijun Liu et al., 2010). A phage display peptide library is a mixture of filamentous phage with foreign peptides on their surface and the coding sequences for the peptides in the viral DNA. Surface display is accomplished by fusing the peptide coding sequence to a coat protein encoding gene either in the minor coat protein cpIII or in the major coat protein cpVIII (Scott and Smith 1990). Each phage clone displays a single peptide, but a library as a whole may represent billions of peptides altogether. The vital advantage of surface exposure is that it allows phage display libraries with vast numbers of peptides to be easily surveyed for clones whose displayed peptides bind specifically to any given molecular target. A number of peptides that bind receptor molecules have been identified using phage display libraries (Natalia et. al., 2002).

None of the compositions or methods available in the art have the potential to restore and or enhance the toxicity of Bt toxins against lepidopteran insect pests such as Pectinophora gossypiella (Pink Bollworm), which have evolved resistance to the Bt toxin, by increasing binding of Cry toxins to insect gut receptors. Thus, there is a need for continuous upgradation and development of novel and improved insecticidal proteins that can specifically target the resistant population of Lepidopteran insect pests.

The present invention overcome the problems of the prior art and deals with modification of Cry1Ac protein using pink bollworm gut binding peptides to enhance its gut binding and eventually activity. More particularly, the present invention provides modified Cry1Ac protein(s) with enhanced insecticidal toxicity with great potential for the production of insect resistant transgenic plants resulting in reduced application of costly and environmentally damaging insecticides, for sustained and environmentally sound agricultural productivity.

### OBJECTS OF THE INVENTION

An important object of the present invention is to provide modified Cry1Ac toxin(s) with enhanced toxicity towards Lepidopteran insect pests particularly resistant Pink Bollworm.

Another important object of the present invention is to provide modified Cry1Ac protein(s) having improved insecticidal activity/toxicity against Lepidopteran insects compared to a native Cry1Ac protein and comprising insertion of one or more peptide sequences at one or more amino acid positions of Cry1Ac.

Another important object of the present invention is to provide a method for identification of site(s) in Cry1Ac where insertions of small peptides can be done without affecting the toxicity.

Another object of the present invention is to design a library of novel gut binding peptides (GBPs) capable of binding to resistant pink bollworm gut.

A further object of the present invention is to provide an insecticidal composition(s) comprising said mutant of Cry1Ac protein(s).

Another object of the present invention is to provide a method for production of insect resistant transgenic plants.

Another object of the present invention is to provide a transgenic plant capable of expressing the mutant Cry1Ac protein(s) and resistant to Lepidopteran insects.

Another object of the present invention is to provide a method of inhibiting growth or killing an insect pest, pest population or resistant pest of the order Lepidoptera.

Another object of the present invention is to provide a method to inhibit pests of the order of Lepidoptera specifically Pink Bollworm or resistant population of Pink Bollworm using said Cry1Ac mutant(s).

### BRIEF DESCRIPTION OF FIGURES

The accompanying figures illustrate some of the embodiments of the present invention and, together with the descriptions, serve to explain the invention. These figures have been provided by way of illustration and not by way of limitation.

FIG. 1 provides a schematic representation for identification of pink bollworm (PBW) gut binding peptides using phage display library.

FIG. 2 shows a scheme of overlap extension PCR for the incorporation of peptides and modification of Cry1Ac protein(s).

FIG. 3 shows a schematic representation of Cry1Ac modifications No. 1 to 16. Each selected peptide was inserted in four insertion sites identified in Cry1Ac coding sequence.

FIG. 4 shows schematic representation of Cry1Ac modifications No. 17 to 19. Peptide AISPSRYFYDET (hereinafter referred to as AIS) was introduced at two, three and four identified insertion sites in Cry1Ac.

FIG. 5 shows schematic representation of Cry1Ac modifications No. 20 to 22. Different modifications were done by combining AIS @ 282 with APTTWFNSDSIT (hereinafter referred to as APT) @ 368, SANYNVQAGWTH (hereinafter referred to as SAN) @ 508 and WAMDGQQHSNNY (hereinafter referred to as WAM) @ 523.

FIG. 6 shows method for collection of Cry1Ac resistant Pink Bollworm from infested field (heavily infested cotton balls and plants) Panel (A): PBW Infested cotton plant, (B): PBW Infested cotton ball with burrowed PBW inside, (C): PBW on cotton ball, (D): Arrangement of cotton balls to harvest live PBW.

FIG. 7 illustrates four insertion sites for peptides in Cry1Ac labelled as 282, 368, 523 and 582.

FIG. 8 shows overlay of native Cry1Ac, Cry1Ac+peptide@282, Cry1Ac+peptide@368, Cry1Ac+peptide@508, Cry1Ac+peptide@523.

FIG. 9 shows results of overlap extension-PCR for peptide insertion. (a) Generation of two different overlapping fragments (b) Joining of two fragments using overlap extension PCR (c) Validation of nucleotide insertion using site specific PCR (d) HRM (High Resolution Melt) profile of native and inserted fragments.

FIG. 10 shows Cry1Ac modifications 1, 5, 9 and 13 (as representation). Four different peptides introduced at the same site (aa282; RG-SAQGIE) and sequence alignments of a region of insertion (nucleotide followed by amino acid) are shown. Inserted nucleotide and peptide sequences are shown in grey background.

FIG. 11 illustrates confirmation of multiple peptide insertion in Cry1Ac. Lane M: Molecular weight marker, Lane 1: Native Cry1Ac (Molecular weight 3537 bp), Lane 2: Cry1Ac with first peptide inserted (Molecular weight 3537+36=3573 bp), Lane 3: Cry1Ac with second peptide inserted (Molecular weight 3537+36+36=3609 bp), Lane 4: Cry1Ac with third peptide inserted (Molecular weight 3537+36+36+36=3645 bp) and Lane 5: Cry1Ac with fourth peptide inserted (Molecular weight 3537+36+36+36+36=3681 bp).

FIG. 12 illustrates the recombinant expression and purification of Native and modified Cry1Ac. Expression of Native Cry1Ac and one modified toxin (SAN @ 282) is shown as representative. Lane M: Molecular weight marker, Lanes 1, 2 and 3: Total protein analysis of bacterial lysate expressing Native Cry1Ac, Lanes 4, 5 and 6: Total protein analysis of bacterial lysate expressing Modified Cry1Ac (SAN @ 282). Lanes 7 and 8: 2 and 4 ug of BSA as standard

FIG. 13 shows the results of Western blot analysis of recombinant Cry1Ac (Native and Modified). Immunoreactivity of native and modified Cry1Ac (SAN @ 282) is shown as representative. M: Molecular weight marker, B1: Untransformed bacterial cell lysate, Nt: Cell lysate of lysate expressing Native Cry1Ac, Lanes (D14, D24, D8 and D13) cell lysates of clones expressing SAN @ 282.

FIG. 14 illustrates the recombinant expression of Cry1Ac incorporated with multiple peptides. Lane M: Molecular weight marker, Lane 1: Native Cry1Ac (predicted molecular weight is 133.12 kDa), Lane 2: Cry1Ac with first peptide inserted (predicted molecular weight 133.12+1.5=134.62 kDa), Lane 3: Cry1Ac with second peptide inserted (predicted molecular weight 133.12+1.5+1.5=135.12kDa), Lane 4: Cry1Ac with third peptide inserted (predicted molecular weight 133.12+1.5+1.5+1.5=136.62 kDa) Lane 5: Cry1Ac with fourth peptide inserted (predicted molecular weight 133.12+1.5+1.5+1.5+1.5=138.12 kDa).

FIG. 15 shows the Mortality of Cry1Ac resistant PBW larvae fed with native and modified Cry1Ac(s).

FIG. 16 shows comparison of growth retardation in PBW larvae fed with native and modified Cry1Ac(s). AD: Artificial Diet only, Native (Native Cry1Ac).

FIG. 17 depicts a binary vector carrying the modified Cry1Ac gene.

FIG. 18 demonstrates the progression from explant selection, Agrobacterium infection, embryogenic callus formation, somatic embryogenesis, and embryo maturation stages, followed by processes like embryo elongation, shoot generation, root formation, primary hardening, and eventual growth of putative transgenic events.

FIG. 19 shows compelling results highlighting the effectiveness of modified toxins in transgenic cotton. (A) demonstrates reduced Pink Bollworm larval survival on leaf discs, while (B) shows visual observations of leaf disc feeding highlighted a significantly reduced consumption in the transgenic events compared to NBt and Bt cotton.

### SUMMARY OF THE INVENTION

The present invention primarily provides modified Cry1Ac protein(s) having enhanced toxicity towards resistant Pink Bollworm (PBW) insect population. More particularly, the present invention relates to identification of specific insertion sites in Cry1Ac and engineering of Cry1Ac(s) by insertion of gut binding peptides into said sites based on in silico studies and modification approach. Insertion of said specific, tightly binding gut peptides in Cry1Ac at one or more identified sites increases the retention of toxin in the PBW gut, resulting in solubilisation and pore formation leading to sepsis and larval death.

The modified Bt toxin(s) exhibit significant toxicity against resistant pink bollworm and thus can be used for generation of insect resistant transgenic plants. More particularly, development of transgenic cotton expressing modified Cry toxins with higher potency, which over the period of time can delay field evolved insect resistance and this will help to sustain Bt cotton technology for a longer duration.

### DETAILED DESCRIPTION OF INVENTION

At the very outset, it may be understood that the ensuing description only illustrates a particular form of this invention. However, such a particular form is only an exemplary embodiment, without intending to imply any limitation on the scope of this invention. Accordingly, the description and examples are to be understood as exemplary embodiments for teaching the invention and not intended to be taken restrictively.

The details of one or more embodiments of the invention are set forth in the accompanying description below including specific details of the best mode contemplated by the inventors for carrying out the invention, by way of example. It will be apparent to one skilled in the art that the present invention may be practiced without limitation to these specific details.

### Abbreviations

Bt—Bacillus thuringiensis
E. coli—Escherichia coli
EDTA—Ethylenediaminetetraacetic acid
GBP—Gut binding peptide
OLE-PCR—Overlap Extension PCR
PBS—Phosphate Buffered Saline
PBW—Pink Bollworm
SDS-PAGE—Sodium dodecyl sulfate polyacrylamide gel electrophoresis
Tris-HCl—Tris Hydrochloride

### Definitions

Unless contraindicated or noted otherwise, throughout this specification, the terms “a” and “an” mean one or more, and the term “or” means and/or.

The use of “comprise”, “comprises”, “comprising”, “contain”, “contains”, “containing”, “include”, “includes”, and “including” are not intended to be limiting. It is to be understood that both the foregoing general description and this detailed description are exemplary and explanatory only and are not restrictive. Wherever there is an indefinite article used, the specification is to be understood as contemplating plurality as well as singularity, unless the context requires otherwise.

Unless otherwise defined, scientific and technical terms used herein shall have the meanings that are commonly understood by those of ordinary skill in the art. Further, unless otherwise required by context, singular terms shall include pluralities and plural terms shall include the singular.

As used herein, biotechnological terms have their conventional meaning as illustrated by the following illustrative definitions:

The term “recombinant” used herein relates to or denotes an organism, cell, or genetic material produced by combining genetic material from more than one origin.

The term “modified” refers to a modified cry protein. The synonyms such as mutant, recombinant, engineered etc. may be used interchangeably and is commonly understood by the person skilled in the art.

The term “insect” refers to insects or pests that feed on Bt cotton plants. Related terms such as pest, pest population or resistant pest are also to be interpreted in a similar manner.

The present invention deals with the modification of Cry1Ac protein using pink bollworm gut binding peptides to enhance its gut binding and eventual insecticidal activity. The inventors of the present invention identified specific site(s) in Cry1Ac coding sequence, where gut binding peptides can be inserted without altering the toxicity of the protein. This was achieved using in silico structural analysis of Cry1Ac protein. Further, the specific and gut binding peptides (GBPs) binding to the resistant Pink Bollworm gut were identified using phage display library. The gut binding peptides frequently appearing in the selected phage population and exhibiting higher phage recovery than other clones were selected. Identified gut binding peptides were then inserted in the specific sites of Cry1Ac identified in the first part, using OLE-PCR. Further to this, the modified toxins with inserted gut binding peptides were recombinantly expressed and partial purification was carried out. Partially purified toxins (both native and modified) were fed to resistant PBW larvae in artificial diets and mortality was observed.

In an important embodiment, the present invention involves identification of specific sites in Cry1Ac protein where modifications/small insertions can be done without altering conformation of protein and toxicity.

In another embodiment, the present invention provides novel gut binding peptides identified using phage display library.

In another important embodiment, the present invention provides modified Cry1Ac protein(s) with enhanced toxicity against field Bt-resistant PBW population.

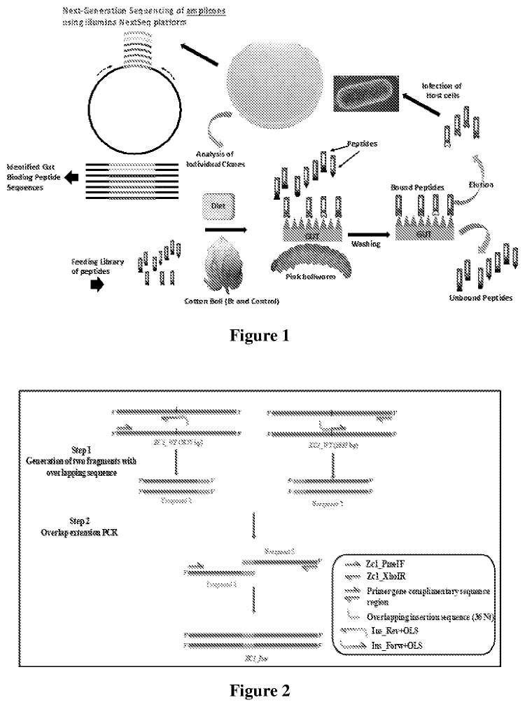

In an embodiment of the present invention, one or more gut binding peptides of 12 amino acid residues selected from SEQ ID NO: 1 to 82 are inserted at at least one of the specific sites selected from amino acid positions 189, 223, 282, 334, 368, 469, 508, 523, 666 and 834 (FIG. 1) of Cry1Ac without affecting the activity and toxicity of the protein.

In a further embodiment, the present invention provides a modified Cry1Ac peptide having insertion of one or more gut binding peptide at two, three and four different sites in Cry1Ac.

An embodiment of the present invention also relates to inhibition of the Lepidopteran pests, specifically Pectinophora gossypiella (Pink Bollworm).

An embodiment of the present invention also relates to extending and using this method for modification of proteins in Bt technology for other related plants not limited to Bt cotton plant.

In an embodiment, the present invention provides a modified Cry1Ac protein comprising one or more gut binding peptide (GBP) sequences selected from SEQ ID NO: 1 to 82, inserted at at least one of the amino acid positions selected from 189, 223, 282, 334, 368, 469, 508, 523, 666 and 834 of Cry1Ac, wherein said mutant Cry1Ac protein has improved insecticidal activity/toxicity against Lepidopteran insects specifically Bt resistant pink bollworm (PBW) compared to native Cry1Ac protein.

In another embodiment of the present invention, the insect is pest, pest population or resistant pest species of Lepidopteran insects specifically pink bollworm (PBW).

In a further embodiment, the present invention provides a method of identifying one or more stable target site(s) in native Cry1Ac protein for insertion of a gut binding peptide comprising:
i. in silico studies comprising structural analysis and insertion of known gut binding peptides to check stability of the protein pre and post insertion; and
ii. identification and selection of stable sites based on the stability results.

In another embodiment, the stable target sites are selected from amino acid positions 189, 223,282, 334, 368, 469, 508, 523, 666 and 834 of Cry1Ac.

In a further embodiment, the present invention provides a method of preparing the modified Cry1Ac protein, comprising:
a) identifying the gut binding peptides (GBPs);
b) in silico identification of one or more stable target site(s) in Cry1Ac protein for insertion of one or more said gut binding peptide(s); and
c) modification of native Cry1Ac gene by insertion of one or more GBPs at at least one or more identified sites to obtain the modified Cry1Ac protein, wherein said modified Cry1Ac protein has improved insecticidal activity/toxicity against Lepidopteran insects specifically Bt resistant pink bollworm (PBW) compared to native Cry1Ac protein.

In another embodiment, the method of identifying gut binding peptides comprises:
i. Screening a phage display library using biopanning protocol;
ii. feeding the insect larvae on the phage display library using either artificial diet or coated cotton balls;
iii. dissecting of larval guts;
iv. elution for bound phages;
v. amplification and enrichment of eluted phages via transfection into host cells for creating a library of peptides;
vi. analysis of individual clones and sequencing; and
vii. shortlisting gut binding peptides that bind to the gut of the insect, wherein the gut binding peptides frequently appearing in the selected phage population and exhibiting higher phage recovery than other clones were selected.

In another embodiment, the present invention provides a polynucleotide comprising a nucleic acid molecule encoding the modified Cry1Ac protein(s) of the present invention.

In yet another embodiment, the present invention provides a vector comprising the polynucleotide of the present invention.

In a further embodiment, the present invention provides a host cell transformed using the vector of the present invention, wherein said cell is capable of expressing modified Cry1Ac(s).

In another embodiment, the host cell is a plant cell.

In yet another embodiment, the present invention provides a method of producing an insect-resistant transgenic plant, comprising:
a. stably integrating the polynucleotide into the genome of a host/plant cell; and
b. obtaining a stably transformed plant from plant/plant cell capable of expressing the modified Cry1Ac, wherein said stably transformed plants are resistant to Lepidopteran insects.

In a further embodiment, the present invention provides a library of gut binding peptides (GBPs) comprising peptides having 12 amino acid residues and defined by SEQ ID NO: 1 to 82 wherein said peptides are capable of binding to one or more gut receptors of Lepidopteran insects.

In another embodiment, the present invention provides an insecticidal formulation comprising an effective amount of the modified Cry1Ac protein(s).

In another embodiment, the present invention provides a composition comprising the insecticidal formulation along with one or more formulating agents selected from but not limited to the group consisting of adjuvants, surfactants, thickeners, stickers, petroleum oils, crop oil concentrate, stabilizing agents, solvents, hygroscopic agents, deposit builders, antifoam agents, buffering agents and activators.

In a further embodiment, the present invention provides a method of inhibiting the growth of an insect pest, insect pest population, or resistant lepidopteran insect pests, comprising contacting said insect pest(s) with the modified Cry1Ac protein.

In yet another embodiment, the present invention provides a method for improving the yield of a crop using the insecticidal formulation.

In another embodiment, the present invention provides designing and construction of binary vector expressing Cry1Ac and its modified versions.

In a further embodiment, the present invention provides generation of transgenic specimens by Agrobacterium-mediated transformation, plant regeneration and the subsequent results of insect feeding bio-assays.

In another embodiment, the present invention provides a transgenic plant cell, plant or plant part carrying the modified Cry1Ac protein of the present invention.

### EXAMPLES

The following examples particularly describe the manner in which the invention is to be performed. But the embodiments disclosed herein do not limit the scope of the invention in any manner.

### Example 1: Identification and Preparation of the Modified Cry1Ac Protein

### 1.1 Identification Gut Binding Peptides (GBPs)

A phage display library containing 12 amino acid residue peptides was screened by using biopanning protocol with resistant pink bollworm larvae (Liu et al., 2010). Larvae were fed on a phage display library using either artificial diet or coated cotton balls. Larval guts were subsequently dissected and eluted for bound phages. Eluted phages were amplified and the enrichment was done by transfecting the host cells to create a library of peptides. The library was screened by either screening of individual clone or by following Next-Gen sequencing. The overall scheme is represented in FIG. 1. Screening of the library generated a list of few hundred peptides containing 12 amino acid residues (36 nucleotides). However, only 84 peptides showing higher abundance were considered.

1.2 In silico Identification of Sites in Cry1Ac Suitable for Peptide Insertion

To identify the sites in Cry1Ac where gut binding peptides can be inserted, in silico studies were carried out. The approach was to randomly insert peptides sequences in different Cry1Ac sites using molecular docking and study the stability of toxin pre and post insertion. Selected peptides (ligands) were processed for docking studies using LigPrep in Schrodinger. Interaction analysis of selected peptides with Cry1Ac model was performed to get insight into exact site(s) and strengths of binding. All ionization states were significantly populated for the pH range 7.0+/−2.0. Desalting was carried out followed by generation of tautomers. ZRANK docking scores were generated and interface salvation energies (kcal/mol) for peptide integrated Cry1Ac studied.

### 1.3 Modification of Cry1Ac Gene by Inserting Identified GBPs

Codon optimisation was done for the obtained peptide sequences for bacterial expression and primers were designed for insertion of these gut binding nucleotides in the selected sites in Cry1Ac. From the list of peptides obtained, four peptides viz. AIS, APT, SAN and WAM were selected initially for cloning purpose. Insertion of 36 nucleotides at a specific site in Cry1Ac coding sequence was achieved using OLE-PCR. Amplification was carried out using specific PCR conditions. Overall scheme of OLE PCR is shown in FIG. 2.

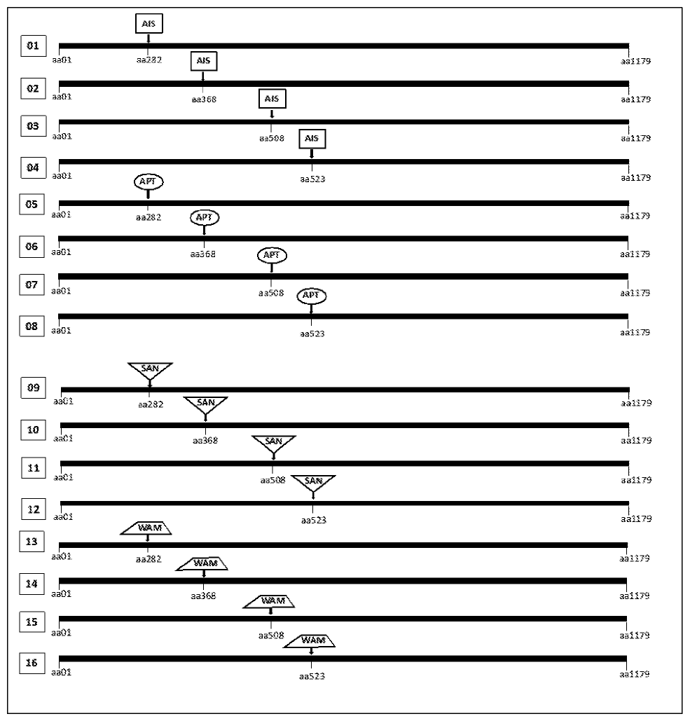

Each selected peptide was inserted at 4 different insertion sites (viz. 282, 368, 508 and 523) generating 16 different modified toxins (Number 1 to 16). The schematic representation of the modifications obtained is shown in FIG. 3. The modified toxins were recombinantly expressed, partially purified and insect feeding bioassays were conducted. From the results obtained, best performing peptide and respective site in Cry1Ac were selected.

### Exemplary Modified Cry1Ac Representative Sequences With Single Insertions (Inserted Peptides Shown in Bold)

(Insertion of peptide AISPSRYFYDET at 189/190)
SEQ ID NO: 83
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGEDA
ATIAISPSRYFYDETNSRYNDLTRLIGNYTDHAVRWYNTGLERVWGPDSRDWIRYNQFRREL
TLTVLDIVSLFPNYDSRTYPIRTVSQLTREIYTNPVLENEDGSFRGSAQGIEGSIRSPHLMD
ILNSITIYTDAHRGEYYWSGHQIMASPVGFSGPEFTFPLYGTMGNAAPQQRIVAQLGQGVYR
TLSSTLYRRPFNIGINNQQLSVLDGTEFAYGTSSNLPSAVYRKSGTVDSLDEIPPQNNNVPP
RQGFSHRLSHVSMFRSGFSNSSVSIIRAPMFSWIHRSAEFNNIIASDSITQIPAVKGNFLEN
GSVISGPGFTGGDLVRLNSSGNNIQNRGYIEVPIHFPSTSTRYRVRVRYASVTPIHLNVNWG
NSSIFSNTVPATATSLDNLQSSDFGYFEGANAFTSSLGDIVGVRNFSGTAGVIIDRFEFIPV
TATLEAEYNLERAQKAVNALFTSTNQLGLKTNVTDYHIDQVSNLVTYLSDEFCLDEKRELSE
KVKHAKRLSDERNLLQDSNEKDINRQPERGWGGSTGITIQGGDDVFKENYVTLSGTFDECYP
TYLYQKIDESKLKAFTRYQLRGYIEDSQDLEIYSIRYNAKHETVNVPGTGSLWPLSAQSPIG
KCGEPNRCAPHLEWNPDLDCSCRDGEKCAHHSHHESLDIDVGCTDLNEDLGVWVIFKIKTQD
GHARLGNLEFLEEKPLVGEALARVKRAEKKWRDKREKLEWETNIVYKEAKESVDALFVNSQY
DQLQADTNIAMIHAADKRVHSIREAYLPELSVIPGVNAAIFEELEGRIFTAFSLYDARNVIK
NGDFNNGLSCWNVKGHVDVEEQNNQRSVLVVPEWEAEVSQEVRVCPGRGYILRVTAYKEGYG
EGCVTIHEIENNTDELKFSNCVEEEIYPNNTVTCNDYTVNQEEYGGAYTSRNRGYNEAPSVP
ADYASVYEEKSYTDGRRENPCELNRGYRDYTPLPVGYVTKELEYFPETDKVWIEIGETEGTF
IVDSVELLLMEE
(Insertion of peptide APTTWFNSDSIT at 223/224)
SEQ ID NO: 84
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGFDA
ATINSRYNDLTRLIGNYTDHAVRWYNTGLERVWGPDSAPTTWENSDSITRDWIRYNQFRREL
TLTVLDIVSLFPNYDSRTYPIRTVSQLTREIYTNPVLENFDGSFRGSAQGIEGSIRSPHLMD
ILNSITIYTDAHRGEYYWSGHQIMASPVGFSGPEFTFPLYGTMGNAAPQQRIVAQLGQGVYR
TLSSTLYRRPENIGINNQQLSVLDGTEFAYGTSSNLPSAVYRKSGTVDSLDEIPPQNNNVPP
RQGFSHRLSHVSMFRSGFSNSSVSIIRAPMFSWIHRSAEFNNIIASDSITQIPAVKGNFLEN
GSVISGPGFTGGDLVRLNSSGNNIQNRGYIEVPIHFPSTSTRYRVRVRYASVTPIHLNVNWG
NSSIFSNTVPATATSLDNLQSSDFGYFEGANAFTSSLGDIVGVRNFSGTAGVIIDRFEFIPV
TATLEAEYNLERAQKAVNALFTSTNQLGLKTNVTDYHIDQVSNLVTYLSDEFCLDEKRELSE
KVKHAKRLSDERNLLQDSNFKDINRQPERGWGGSTGITIQGGDDVFKENYVTLSGTFDECYP
TYLYQKIDESKLKAFTRYQLRGYIEDSQDLEIYSIRYNAKHETVNVPGTGSLWPLSAQSPIG
KCGEPNRCAPHLEWNPDLDCSCRDGEKCAHHSHHFSLDIDVGCTDLNEDLGVWVIFKIKTQD
GHARLGNLEFLEEKPLVGEALARVKRAEKKWRDKREKLEWETNIVYKEAKESVDALFVNSQY
DQLQADTNIAMIHAADKRVHSIREAYLPELSVIPGVNAAIFEELEGRIFTAFSLYDARNVIK
NGDFNNGLSCWNVKGHVDVEEQNNQRSVLVVPEWEAEVSQEVRVCPGRGYILRVTAYKEGYG
EGCVTIHEIENNTDELKFSNCVEEEIYPNNTVTCNDYTVNQEEYGGAYTSRNRGYNEAPSVP
ADYASVYEEKSYTDGRRENPCELNRGYRDYTPLPVGYVTKELEYFPETDKVWIEIGETEGTE
IVDSVELLLMEE
(Insertion of peptide MPLRYPTQVTLD at 282/283)
SEQ ID NO: 85
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGEDA
ATINSRYNDLTRLIGNYTDHAVRWYNTGLERVWGPDSRDWIRYNQFRRELTLTVLDIVSLFP
NYDSRTYPIRTVSQLTREIYTNPVLENEDGSFRGMPLRYPTQVTLDSAQGIEGSIRSPHLMD
ILNSITIYTDAHRGEYYWSGHQIMASPVGESGPEFTFPLYGTMGNAAPQQRIVAQLGQGVYR
TLSSTLYRRPFNIGINNQQLSVLDGTEFAYGTSSNLPSAVYRKSGTVDSLDEIPPQNNNVPP
RQGFSHRLSHVSMFRSGFSNSSVSIIRAPMESWIHRSAEENNIIASDSITQIPAVKGNFLEN
GSVISGPGFTGGDLVRLNSSGNNIQNRGYIEVPIHFPSTSTRYRVRVRYASVTPIHLNVNWG
NSSIFSNTVPATATSLDNLQSSDFGYFEGANAFTSSLGDIVGVRNFSGTAGVIIDRFEFIPV
TATLEAEYNLERAQKAVNALFTSTNQLGLKTNVTDYHIDQVSNLVTYLSDEFCLDEKRELSE
KVKHAKRLSDERNLLQDSNFKDINRQPERGWGGSTGITIQGGDDVFKENYVTLSGTFDECYP
TYLYQKIDESKLKAFTRYQLRGYIEDSQDLEIYSIRYNAKHETVNVPGTGSLWPLSAQSPIG
KCGEPNRCAPHLEWNPDLDCSCRDGEKCAHHSHHFSLDIDVGCTDLNEDLGVWVIFKIKTQD
GHARLGNLEFLEEKPLVGEALARVKRAEKKWRDKREKLEWETNIVYKEAKESVDALFVNSQY
DQLQADTNIAMIHAADKRVHSIREAYLPELSVIPGVNAAIFEELEGRIFTAFSLYDARNVIK
NGDFNNGLSCWNVKGHVDVEEQNNQRSVLVVPEWEAEVSQEVRVCPGRGYILRVTAYKEGYG
EGCVTIHEIENNTDELKFSNCVEEEIYPNNTVTCNDYTVNQEEYGGAYTSRNRGYNEAPSVP
ADYASVYEEKSYTDGRRENPCELNRGYRDYTPLPVGYVTKELEYFPETDKVWIEIGETEGTF
IVDSVELLLMEE
(Insertion of peptide SANYNVQAGWTH at 334/335)
SEQ ID NO: 86
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGEDA
ATINSRYNDLTRLIGNYTDHAVRWYNTGLERVWGPDSRDWIRYNQFRRELTLTVLDIVSLEP
NYDSRTYPIRTVSQLTREIYTNPVLENEDGSFRGSAQGIEGSIRSPHLMDILNSITIYTDAH
RGEYYWSGHQIMASPVGFSGPEFTSANYNVQAGWTHFPLYGTMGNAAPQQRIVAQLGQGVYR
TLSSTLYRRPFNIGINNQQLSVLDGTEFAYGTSSNLPSAVYRKSGTVDSLDEIPPQNNNVPP
RQGFSHRLSHVSMFRSGFSNSSVSIIRAPMFSWIHRSAEFNNIIASDSITQIPAVKGNFLEN
GSVISGPGFTGGDLVRLNSSGNNIQNRGYIEVPIHFPSTSTRYRVRVRYASVTPIHLNVNWG
NSSIFSNTVPATATSLDNLQSSDEGYFEGANAFTSSLGDIVGVRNFSGTAGVIIDRFEFIPV
TATLEAEYNLERAQKAVNALFTSTNQLGLKTNVTDYHIDQVSNLVTYLSDEFCLDEKRELSE
KVKHAKRLSDERNLLQDSNFKDINRQPERGWGGSTGITIQGGDDVFKENYVTLSGTFDECYP
TYLYQKIDESKLKAFTRYQLRGYIEDSQDLEIYSIRYNAKHETVNVPGTGSLWPLSAQSPIG
KCGEPNRCAPHLEWNPDLDCSCRDGEKCAHHSHHFSLDIDVGCTDLNEDLGVWVIFKIKTQD
GHARLGNLEFLEEKPLVGEALARVKRAEKKWRDKREKLEWETNIVYKEAKESVDALFVNSQY
DQLQADTNIAMIHAADKRVHSIREAYLPELSVIPGVNAAIFEELEGRIFTAFSLYDARNVIK
NGDFNNGLSCWNVKGHVDVEEQNNQRSVLVVPEWEAEVSQEVRVCPGRGYILRVTAYKEGYG
EGCVTIHEIENNTDELKESNCVEEEIYPNNTVTCNDYTVNQEEYGGAYTSRNRGYNEAPSVP
ADYASVYEEKSYTDGRRENPCELNRGYRDYTPLPVGYVTKELEYFPETDKVWIEIGETEGTF
IVDSVELLLMEE
(Insertion of peptide DGSMLNRMRGFS at 368/369)
SEQ ID NO: 87
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGFDA
ATINSRYNDLTRLIGNYTDHAVRWYNTGLERVWGPDSRDWIRYNQFRRELTLTVLDIVSLEP
NYDSRTYPIRTVSQLTREIYTNPVLENFDGSFRGSAQGIEGSIRSPHLMDILNSITIYTDAH
RGEYYWSGHQIMASPVGFSGPEFTFPLYGTMGNAAPQQRIVAQLGQGVYRTLSSTLYRDGSM
LNRMRGFSRPENIGINNQQLSVLDGTEFAYGTSSNLPSAVYRKSGTVDSLDEIPPQNNNVPP
RQGFSHRLSHVSMFRSGFSNSSVSIIRAPMFSWIHRSAEFNNIIASDSITQIPAVKGNELEN
GSVISGPGFTGGDLVRLNSSGNNIQNRGYIEVPIHFPSTSTRYRVRVRYASVTPIHLNVNWG
NSSIFSNTVPATATSLDNLQSSDFGYFEGANAFTSSLGDIVGVRNFSGTAGVIIDRFEFIPV
TATLEAEYNLERAQKAVNALFTSTNQLGLKTNVTDYHIDQVSNLVTYLSDEFCLDEKRELSE
KVKHAKRLSDERNLLQDSNEKDINRQPERGWGGSTGITIQGGDDVFKENYVTLSGTEDECYP
TYLYQKIDESKLKAFTRYQLRGYIEDSQDLEIYSIRYNAKHETVNVPGTGSLWPLSAQSPIG
KCGEPNRCAPHLEWNPDLDCSCRDGEKCAHHSHHFSLDIDVGCTDLNEDLGVWVIFKIKTQD
GHARLGNLEFLEEKPLVGEALARVKRAEKKWRDKREKLEWETNIVYKEAKESVDALFVNSQY
DQLQADTNIAMIHAADKRVHSIREAYLPELSVIPGVNAAIFEELEGRIFTAFSLYDARNVIK
NGDFNNGLSCWNVKGHVDVEEQNNQRSVLVVPEWEAEVSQEVRVCPGRGYILRVTAYKEGYG
EGCVTIHEIENNTDELKFSNCVEEEIYPNNTVTCNDYTVNQEEYGGAYTSRNRGYNEAPSVP
ADYASVYEEKSYTDGRRENPCELNRGYRDYTPLPVGYVTKELEYFPETDKVWIEIGETEGTF
IVDSVELLLMEE
(Insertion of peptide WAMDGQQHSNNY at 479/480)
SEQ ID NO: 88
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGFDA
ATINSRYNDLTRLIGNYTDHAVRWYNTGLERVWGPDSRDWIRYNQFRRELTLTVLDIVSLEP
NYDSRTYPIRTVSQLTREIYTNPVLENEDGSFRGSAQGIEGSIRSPHLMDILNSITIYTDAH
RGEYYWSGHQIMASPVGFSGPEFTFPLYGTMGNAAPQQRIVAQLGQGVYRTLSSTLYRRPEN
IGINNQQLSVLDGTEFAYGTSSNLPSAVYRKSGTVDSLDEIPPQNNNVPPRQGFSHRLSHVS
MERSGFSNSSVSIIRAPMESWIHRSAEFNNIIASDSITQIPAVKGWAMDGQQHSNNYNFLEN
GSVISGPGFTGGDLVRLNSSGNNIQNRGYIEVPIHFPSTSTRYRVRVRYASVTPIHLNVNWG
NSSIFSNTVPATATSLDNLQSSDFGYFEGANAFTSSLGDIVGVRNFSGTAGVIIDRFEFIPV
TATLEAEYNLERAQKAVNALFTSTNQLGLKTNVTDYHIDQVSNLVTYLSDEFCLDEKRELSE
KVKHAKRLSDERNLLQDSNFKDINRQPERGWGGSTGITIQGGDDVFKENYVTLSGTFDECYP
TYLYQKIDESKLKAFTRYQLRGYIEDSQDLEIYSIRYNAKHETVNVPGTGSLWPLSAQSPIG
KCGEPNRCAPHLEWNPDLDCSCRDGEKCAHHSHHFSLDIDVGCTDLNEDLGVWVIFKIKTQD
GHARLGNLEFLEEKPLVGEALARVKRAEKKWRDKREKLEWETNIVYKEAKESVDALFVNSQY
DQLQADTNIAMIHAADKRVHSIREAYLPELSVIPGVNAAIFEELEGRIFTAFSLYDARNVIK
NGDFNNGLSCWNVKGHVDVEEQNNQRSVLVVPEWEAEVSQEVRVCPGRGYILRVTAYKEGYG
EGCVTIHEIENNTDELKFSNCVEEEIYPNNTVTCNDYTVNQEEYGGAYTSRNRGYNEAPSVP
ADYASVYEEKSYTDGRRENPCELNRGYRDYTPLPVGYVTKELEYFPETDKVWIEIGETEGTF
IVDSVELLLMEE
(Insertion of peptide EPELDNARVLQI at 508/509)
SEQ ID NO: 89
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGEDA
ATINSRYNDLTRLIGNYTDHAVRWYNTGLERVWGPDSRDWIRYNQFRRELTLTVLDIVSLFP
NYDSRTYPIRTVSQLTREIYTNPVLENFDGSFRGSAQGIEGSIRSPHLMDILNSITIYTDAH
RGEYYWSGHQIMASPVGFSGPEFTFPLYGTMGNAAPQQRIVAQLGQGVYRTLSSTLYRRPEN
IGINNQQLSVLDGTEFAYGTSSNLPSAVYRKSGTVDSLDEIPPQNNNVPPRQGFSHRLSHVS
MFRSGFSNSSVSIIRAPMESWIHRSAEENNIIASDSITQIPAVKGNFLENGSVISGPGFTGG
DLVRLNSSGNNIEPELDNARVLQIQNRGYIEVPIHFPSTSTRYRVRVRYASVTPIHLNVNWG
NSSIFSNTVPATATSLDNLQSSDFGYFEGANAFTSSLGDIVGVRNFSGTAGVIIDRFEFIPV
TATLEAEYNLERAQKAVNALFTSTNQLGLKTNVTDYHIDQVSNLVTYLSDEFCLDEKRELSE
KVKHAKRLSDERNLLQDSNFKDINRQPERGWGGSTGITIQGGDDVFKENYVTLSGTFDECYP
TYLYQKIDESKLKAFTRYQLRGYIEDSQDLEIYSIRYNAKHETVNVPGTGSLWPLSAQSPIG
KCGEPNRCAPHLEWNPDLDCSCRDGEKCAHHSHHFSLDIDVGCTDLNEDLGVWVIFKIKTQD
GHARLGNLEFLEEKPLVGEALARVKRAEKKWRDKREKLEWETNIVYKEAKESVDALFVNSQY
DQLQADTNIAMIHAADKRVHSIREAYLPELSVIPGVNAAIFEELEGRIFTAFSLYDARNVIK
NGDFNNGLSCWNVKGHVDVEEQNNQRSVLVVPEWEAEVSQEVRVCPGRGYILRVTAYKEGYG
EGCVTIHEIENNTDELKFSNCVEEEIYPNNTVTCNDYTVNQEEYGGAYTSRNRGYNEAPSVP
ADYASVYEEKSYTDGRRENPCELNRGYRDYTPLPVGYVTKELEYFPETDKVWIEIGETEGTF
IVDSVELLLMEE
(Insertion of peptide APTTWFNSDSIT at 523/524)
SEQ ID NO: 90
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGEDA
ATINSRYNDLTRLIGNYTDHAVRWYNTGLERVWGPDSRDWIRYNQFRRELTLTVLDIVSLFP
NYDSRTYPIRTVSQLTREIYTNPVLENFDGSFRGSAQGIEGSIRSPHLMDILNSITIYTDAH
RGEYYWSGHQIMASPVGFSGPEFTFPLYGTMGNAAPQQRIVAQLGQGVYRTLSSTLYRRPEN
IGINNQQLSVLDGTEFAYGTSSNLPSAVYRKSGTVDSLDEIPPQNNNVPPRQGFSHRLSHVS
MFRSGFSNSSVSIIRAPMESWIHRSAEFNNIIASDSITQIPAVKGNFLENGSVISGPGFTGG
DLVRLNSSGNNIQNRGYIEVPIHFPSTAPTTWFNSDSITSTRYRVRVRYASVTPIHLNVNWG
NSSIFSNTVPATATSLDNLQSSDFGYFEGANAFTSSLGDIVGVRNFSGTAGVIIDRFEFIPV
TATLEAEYNLERAQKAVNALFTSTNQLGLKTNVTDYHIDQVSNLVTYLSDEFCLDEKRELSE
KVKHAKRLSDERNLLQDSNFKDINRQPERGWGGSTGITIQGGDDVFKENYVTLSGTFDECYP
TYLYQKIDESKLKAFTRYQLRGYIEDSQDLEIYSIRYNAKHETVNVPGTGSLWPLSAQSPIG
KCGEPNRCAPHLEWNPDLDCSCRDGEKCAHHSHHFSLDIDVGCTDLNEDLGVWVIFKIKTQD
GHARLGNLEFLEEKPLVGEALARVKRAEKKWRDKREKLEWETNIVYKEAKESVDALFVNSQY
DQLQADTNIAMIHAADKRVHSIREAYLPELSVIPGVNAAIFEELEGRIFTAFSLYDARNVIK
NGDFNNGLSCWNVKGHVDVEEQNNQRSVLVVPEWEAEVSQEVRVCPGRGYILRVTAYKEGYG
EGCVTIHEIENNTDELKESNCVEEEIYPNNTVTCNDYTVNQEEYGGAYTSRNRGYNEAPSVP
ADYASVYEEKSYTDGRRENPCELNRGYRDYTPLPVGYVTKELEYFPETDKVWIEIGETEGTF
IVDSVELLLMEE
(Insertion of peptide APTTWFNSDSIT at 666/667)
SEQ ID NO: 91
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGFDA
ATINSRYNDLTRLIGNYTDHAVRWYNTGLERVWGPDSRDWIRYNQFRRELTLTVLDIVSLFP
NYDSRTYPIRTVSQLTREIYTNPVLENFDGSFRGSAQGIEGSIRSPHLMDILNSITIYTDAH
RGEYYWSGHQIMASPVGFSGPEFTFPLYGTMGNAAPQQRIVAQLGQGVYRTLSSTLYRRPEN
IGINNQQLSVLDGTEFAYGTSSNLPSAVYRKSGTVDSLDEIPPQNNNVPPRQGFSHRLSHVS
MFRSGFSNSSVSIIRAPMFSWIHRSAEFNNIIASDSITQIPAVKGNFLENGSVISGPGFTGG
DLVRLNSSGNNIQNRGYIEVPIHFPSTSTRYRVRVRYASVTPIHLNVNWGNSSIFSNTVPAT
ATSLDNLQSSDFGYFEGANAFTSSLGDIVGVRNFSGTAGVIIDRFEFIPVTATLEAEYNLER
AQKAVNALFTSTNQLGLKTNVTDYHIDQVSNLVTYLSDEFCLDEKRAPTTWFNSDSITELSE
KVKHAKRLSDERNLLQDSNFKDINRQPERGWGGSTGITIQGGDDVFKENYVTLSGTFDECYP
TYLYQKIDESKLKAFTRYQLRGYIEDSQDLEIYSIRYNAKHETVNVPGTGSLWPLSAQSPIG
KCGEPNRCAPHLEWNPDLDCSCRDGEKCAHHSHHFSLDIDVGCTDLNEDLGVWVIFKIKTQD
GHARLGNLEFLEEKPLVGEALARVKRAEKKWRDKREKLEWETNIVYKEAKESVDALFVNSQY
DQLQADTNIAMIHAADKRVHSIREAYLPELSVIPGVNAAIFEELEGRIFTAFSLYDARNVIK
NGDFNNGLSCWNVKGHVDVEEQNNQRSVLVVPEWEAEVSQEVRVCPGRGYILRVTAYKEGYG
EGCVTIHEIENNTDELKESNCVEEEIYPNNTVTCNDYTVNQEEYGGAYTSRNRGYNEAPSVP
ADYASVYEEKSYTDGRRENPCELNRGYRDYTPLPVGYVTKELEYFPETDKVWIEIGETEGTF
IVDSVELLLMEE
(Insertion of peptide WAMDGQQHSNNY at 834/835)
SEQ ID NO: 92
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGFDA
ATINSRYNDLTRLIGNYTDHAVRWYNTGLERVWGPDSRDWIRYNQFRRELTLTVLDIVSLFP
NYDSRTYPIRTVSQLTREIYTNPVLENFDGSFRGSAQGIEGSIRSPHLMDILNSITIYTDAH
RGEYYWSGHQIMASPVGFSGPEFTFPLYGTMGNAAPQQRIVAQLGQGVYRTLSSTLYRRPEN
IGINNQQLSVLDGTEFAYGTSSNLPSAVYRKSGTVDSLDEIPPQNNNVPPRQGFSHRLSHVS
MFRSGFSNSSVSIIRAPMFSWIHRSAEFNNIIASDSITQIPAVKGNFLENGSVISGPGFTGG
DLVRLNSSGNNIQNRGYIEVPIHFPSTSTRYRVRVRYASVTPIHLNVNWGNSSIFSNTVPAT
ATSLDNLQSSDFGYFEGANAFTSSLGDIVGVRNFSGTAGVIIDRFEFIPVTATLEAEYNLER
AQKAVNALFTSTNQLGLKTNVTDYHIDQVSNLVTYLSDEFCLDEKRELSEKVKHAKRLSDER
NLLQDSNFKDINRQPERGWGGSTGITIQGGDDVFKENYVTLSGTFDECYPTYLYQKIDESKL
KAFTRYQLRGYIEDSQDLEIYSIRYNAKHETVNVPGTGSLWPLSAQSPIGKCGEPNRCAPHL
EWNPDLDCSCRDGEKCAHHSHHFSLDIDWAMDGQQHSNNYVGCTDLNEDLGVWVIFKIKTQD
GHARLGNLEFLEEKPLVGEALARVKRAEKKWRDKREKLEWETNIVYKEAKESVDALFVNSQY
DQLQADTNIAMIHAADKRVHSIREAYLPELSVIPGVNAAIFEELEGRIFTAFSLYDARNVIK
NGDFNNGLSCWNVKGHVDVEEQNNQRSVLVVPEWEAEVSQEVRVCPGRGYILRVTAYKEGYG
EGCVTIHEIENNTDELKESNCVEEEIYPNNTVTCNDYTVNQEEYGGAYTSRNRGYNEAPSVP
ADYASVYEEKSYTDGRRENPCELNRGYRDYTPLPVGYVTKELEYFPETDKVWIEIGETEGTF
IVDSVELLLMEE

1.4 Modification of Cry1Ac with Multiple Insertions of Peptides

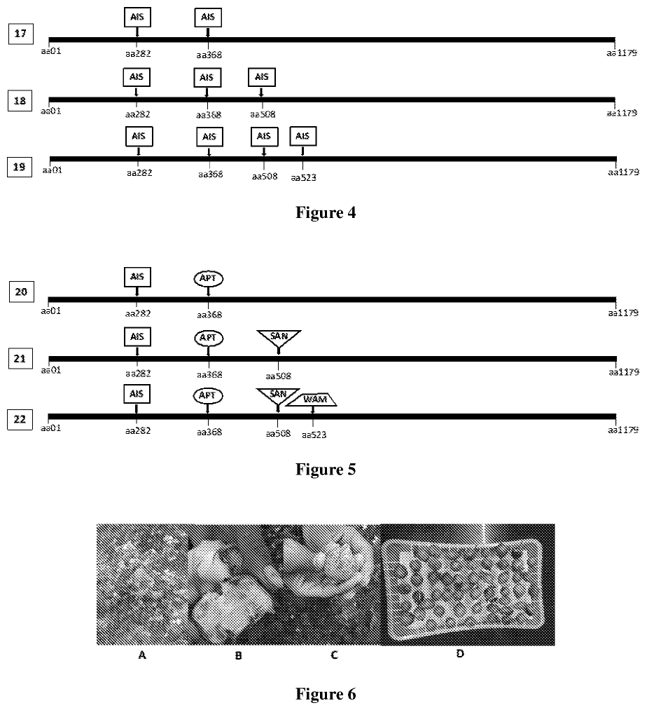

It was hypothesized that insertion of more copies of the peptides could enhance binding to the gut leading to more toxicity. Insertion of the peptide AIS was carried out at two, three and four different sites in Cry1Ac and modifications (No. 17 to No. 19) were done, as shown in FIG. 4. Multiple insertions of peptides were also carried out using molecular cloning techniques as discussed earlier. Peptides were added one at a time at the desired site of insertion. Successful insertion of multiple peptides was confirmed by gel shift pattern as well as DNA sequencing. The modified Cry1Acs with double, triple and quadruple insertion of AIS along with previously used single insertion were recombinantly expressed, partially purified and insect feeding bioassays were carried out.

As introduction of same peptide in more than one site did not show any further enhancement in the activity or toxicity, best performing peptide-insertion site combination (from initial 16 modifications) was explored by the inventors. From the feeding bioassays data, best modification with a combination of peptide and its respective site was selected viz. APT at position 368, SAN at position 508 and WAM at position 523. These modifications were combined with the first successful modification with AIS at position 282 to create modifications No. 20 to 22 as illustrated in FIG. 5.

### Exemplary Modified Cry1Ac Representative Sequences with Multiple Peptide Insertions (Inserted Peptides Shown in Bold)

(Insertion of peptide APTTWFNSDSIT at 282/283 and 368/369)
SEQ ID NO: 93
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGFDA
ATIAPTTWFNSDSITNSRYNDLTRLIGNYTDHAVRWYNTGLERVWGPDSRDWIRYNQFRREL
TLTVLDIVSLFPNYDSRTYPIRTVSQLTREIYTNPVLENFDGSFRGSAQGIEGSIRSPHLMD
ILNSITIYTDAHRGEYYWSGHQIMASPVGFSGPEFTFPLYGTMGNAAPQQRIVAQLGQGVYR
TLSSTLYRAPTTWFNSDSITRPFNIGINNQQLSVLDGTEFAYGTSSNLPSAVYRKSGTVDSL
DEIPPQNNNVPPRQGFSHRLSHVSMFRSGESNSSVSIIRAPMFSWIHRSAEENNIIASDSIT
QIPAVKGNFLFNGSVISGPGFTGGDLVRLNSSGNNIQNRGYIEVPIHFPSTSTRYRVRVRYA
SVTPIHLNVNWGNSSIFSNTVPATATSLDNLQSSDEGYFEGANAFTSSLGDIVGVRNFSGTA
GVIIDRFEFIPVTATLEAEYNLERAQKAVNALFTSTNQLGLKTNVTDYHIDQVSNLVTYLSD
EFCLDEKRELSEKVKHAKRLSDERNLLQDSNFKDINRQPERGWGGSTGITIQGGDDVFKENY
VTLSGTFDECYPTYLYQKIDESKLKAFTRYQLRGYIEDSQDLEIYSIRYNAKHETVNVPGTG
SLWPLSAQSPIGKCGEPNRCAPHLEWNPDLDCSCRDGEKCAHHSHHFSLDIDVGCTDLNEDL
GVWVIFKIKTQDGHARLGNLEFLEEKPLVGEALARVKRAEKKWRDKREKLEWETNIVYKEAK
ESVDALFVNSQYDQLQADTNIAMIHAADKRVHSIREAYLPELSVIPGVNAAIFEELEGRIFT
AFSLYDARNVIKNGDENNGLSCWNVKGHVDVEEQNNQRSVLVVPEWEAEVSQEVRVCPGRGY
ILRVTAYKEGYGEGCVTIHEIENNTDELKESNCVEEEIYPNNTVTCNDYTVNQEEYGGAYTS
RNRGYNEAPSVPADYASVYEEKSYTDGRRENPCELNRGYRDYTPLPVGYVTKELEYFPETDK
VWIEIGETEGTFIVDSVELLLMEE
(Insertion of peptide APTTWFNSDSIT at 282/283, 368/369 and 508/509)
SEQ ID NO: 94
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGEDA
ATIAPTTWFNSDSITNSRYNDLTRLIGNYTDHAVRWYNTGLERVWGPDSRDWIRYNQFRREL
TLTVLDIVSLFPNYDSRTYPIRTVSQLTREIYTNPVLENFDGSFRGSAQGIEGSIRSPHLMD
ILNSITIYTDAHRGEYYWSGHQIMASPVGFSGPEFTFPLYGTMGNAAPQQRIVAQLGQGVYR
TLSSTLYRAPTTWFNSDSITRPFNIGINNQQLSVLDGTEFAYGTSSNLPSAVYRKSGTVDSL
DEIPPQNNNVPPRQGFSHRLSHVSMFRSGFSNSSVSIIRAPMFSWIHRSAEFNNIIASDSIT
QIPAVKGNFLENGSVISGPGFTGGDLVRLNSSGNNIAPTTWFNSDSITQNRGYIEVPIHFPS
TSTRYRVRVRYASVTPIHLNVNWGNSSIFSNTVPATATSLDNLQSSDFGYFEGANAFTSSLG
DIVGVRNFSGTAGVIIDRFEFIPVTATLEAEYNLERAQKAVNALFTSTNQLGLKTNVTDYHI
DQVSNLVTYLSDEFCLDEKRELSEKVKHAKRLSDERNLLQDSNEKDINRQPERGWGGSTGIT
IQGGDDVFKENYVTLSGTFDECYPTYLYQKIDESKLKAFTRYQLRGYIEDSQDLEIYSIRYN
AKHETVNVPGTGSLWPLSAQSPIGKCGEPNRCAPHLEWNPDLDCSCRDGEKCAHHSHHFSLD
IDVGCTDLNEDLGVWVIFKIKTQDGHARLGNLEFLEEKPLVGEALARVKRAEKKWRDKREKL
EWETNIVYKEAKESVDALFVNSQYDQLQADTNIAMIHAADKRVHSIREAYLPELSVIPGVNA
AIFEELEGRIFTAFSLYDARNVIKNGDENNGLSCWNVKGHVDVEEQNNQRSVLVVPEWEAEV
SQEVRVCPGRGYILRVTAYKEGYGEGCVTIHEIENNTDELKFSNCVEEEIYPNNTVTCNDYT
VNQEEYGGAYTSRNRGYNEAPSVPADYASVYEEKSYTDGRRENPCELNRGYRDYTPLPVGYV
TKELEYFPETDKVWIEIGETEGTFIVDSVELLLMEE
(Insertion of same peptide at 282/283, 368/369, 508/509
and 523/524)
SEQ ID NO: 95
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGEDA
ATIAPTTWFNSDSITNSRYNDLTRLIGNYTDHAVRWYNTGLERVWGPDSRDWIRYNQFRREL
TLTVLDIVSLFPNYDSRTYPIRTVSQLTREIYTNPVLENEDGSERGSAQGIEGSIRSPHLMD
ILNSITIYTDAHRGEYYWSGHQIMASPVGFSGPEFTFPLYGTMGNAAPQQRIVAQLGQGVYR
TLSSTLYRAPTTWFNSDSITRPFNIGINNQQLSVLDGTEFAYGTSSNLPSAVYRKSGTVDSL
DEIPPQNNNVPPRQGFSHRLSHVSMFRSGFSNSSVSIIRAPMFSWIHRSAEFNNIIASDSIT
QIPAVKGNFLFNGSVISGPGFTGGDLVRLNSSGNNIAPTTWFNSDSITQNRGYIEVPIHFPS
TAPTTWFNSDSITSTRYRVRVRYASVTPIHLNVNWGNSSIFSNTVPATATSLDNLQSSDFGY
FEGANAFTSSLGDIVGVRNFSGTAGVIIDRFEFIPVTATLEAEYNLERAQKAVNALFTSTNQ
LGLKTNVTDYHIDQVSNLVTYLSDEFCLDEKRELSEKVKHAKRLSDERNLLQDSNEKDINRQ
PERGWGGSTGITIQGGDDVFKENYVTLSGTFDECYPTYLYQKIDESKLKAFTRYQLRGYIED
SQDLEIYSIRYNAKHETVNVPGTGSLWPLSAQSPIGKCGEPNRCAPHLEWNPDLDCSCRDGE
KCAHHSHHFSLDIDVGCTDLNEDLGVWVIFKIKTQDGHARLGNLEFLEEKPLVGEALARVKR
AEKKWRDKREKLEWETNIVYKEAKESVDALFVNSQYDQLQADTNIAMIHAADKRVHSIREAY
LPELSVIPGVNAAIFEELEGRIFTAFSLYDARNVIKNGDENNGLSCWNVKGHVDVEEQNNQR
SVLVVPEWEAEVSQEVRVCPGRGYILRVTAYKEGYGEGCVTIHEIENNTDELKFSNCVEEEI
YPNNTVTCNDYTVNQEEYGGAYTSRNRGYNEAPSVPADYASVYEEKSYTDGRRENPCELNRG
YRDYTPLPVGYVTKELEYFPETDKVWIEIGETEGTFIVDSVELLLMEE
(Insertion of different peptides at 282/283 and 368/369)
SEQ ID NO: 96
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGEDA
ATIMPLRYPTQVTLDNSRYNDLTRLIGNYTDHAVRWYNTGLERVWGPDSRDWIRYNQFRREL
TLTVLDIVSLFPNYDSRTYPIRTVSQLTREIYTNPVLENEDGSFRGSAQGIEGSIRSPHLMD
ILNSITIYTDAHRGEYYWSGHQIMASPVGFSGPEFTFPLYGTMGNAAPQQRIVAQLGQGVYR
TLSSTLYREPELDNARVLQIRPFNIGINNQQLSVLDGTEFAYGTSSNLPSAVYRKSGTVDSL
DEIPPQNNNVPPRQGFSHRLSHVSMERSGFSNSSVSIIRAPMFSWIHRSAEFNNIIASDSIT
QIPAVKGNFLENGSVISGPGFTGGDLVRLNSSGNNIQNRGYIEVPIHFPSTSTRYRVRVRYA
SVTPIHLNVNWGNSSIFSNTVPATATSLDNLQSSDFGYFEGANAFTSSLGDIVGVRNFSGTA
GVIIDRFEFIPVTATLEAEYNLERAQKAVNALFTSTNQLGLKTNVTDYHIDQVSNLVTYLSD
EFCLDEKRELSEKVKHAKRLSDERNLLQDSNFKDINRQPERGWGGSTGITIQGGDDVFKENY
VTLSGTFDECYPTYLYQKIDESKLKAFTRYQLRGYIEDSQDLEIYSIRYNAKHETVNVPGTG
SLWPLSAQSPIGKCGEPNRCAPHLEWNPDLDCSCRDGEKCAHHSHHFSLDIDVGCTDLNEDL
GVWVIFKIKTQDGHARLGNLEFLEEKPLVGEALARVKRAEKKWRDKREKLEWETNIVYKEAK
ESVDALFVNSQYDQLQADTNIAMIHAADKRVHSIREAYLPELSVIPGVNAAIFEELEGRIFT
AFSLYDARNVIKNGDENNGLSCWNVKGHVDVEEQNNQRSVLVVPEWEAEVSQEVRVCPGRGY
ILRVTAYKEGYGEGCVTIHEIENNTDELKESNCVEEEIYPNNTVTCNDYTVNQEEYGGAYTS
RNRGYNEAPSVPADYASVYEEKSYTDGRRENPCELNRGYRDYTPLPVGYVTKELEYFPETDK
VWIEIGETEGTFIVDSVELLLMEE
(Insertion of three different peptides at 282/283, 368/369
and 508/509)
SEQ ID NO: 97
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGEDA
ATIMPLRYPTQVTLDNSRYNDLTRLIGNYTDHAVRWYNTGLERVWGPDSRDWIRYNQFRREL
TLTVLDIVSLFPNYDSRTYPIRTVSQLTREIYTNPVLENFDGSFRGSAQGIEGSIRSPHLMD
ILNSITIYTDAHRGEYYWSGHQIMASPVGFSGPEFTFPLYGTMGNAAPQQRIVAQLGQGVYR
TLSSTLYREPELDNARVLQIRPFNIGINNQQLSVLDGTEFAYGTSSNLPSAVYRKSGTVDSL
DEIPPQNNNVPPRQGFSHRLSHVSMFRSGFSNSSVSIIRAPMFSWIHRSAEFNNIIASDSIT
QIPAVKGNFLENGSVISGPGFTGGDLVRLNSSGNNIMADQTMGLHHGMQNRGYIEVPIHFPS
TSTRYRVRVRYASVTPIHLNVNWGNSSIFSNTVPATATSLDNLQSSDFGYFEGANAFTSSLG
DIVGVRNFSGTAGVIIDRFEFIPVTATLEAEYNLERAQKAVNALFTSTNQLGLKTNVTDYHI
DQVSNLVTYLSDEFCLDEKRELSEKVKHAKRLSDERNLLQDSNFKDINRQPERGWGGSTGIT
IQGGDDVFKENYVTLSGTFDECYPTYLYQKIDESKLKAFTRYQLRGYIEDSQDLEIYSIRYN
AKHETVNVPGTGSLWPLSAQSPIGKCGEPNRCAPHLEWNPDLDCSCRDGEKCAHHSHHESLD
IDVGCTDLNEDLGVWVIFKIKTQDGHARLGNLEFLEEKPLVGEALARVKRAEKKWRDKREKL
EWETNIVYKEAKESVDALFVNSQYDQLQADTNIAMIHAADKRVHSIREAYLPELSVIPGVNA
AIFEELEGRIFTAFSLYDARNVIKNGDENNGLSCWNVKGHVDVEEQNNQRSVLVVPEWEAEV
SQEVRVCPGRGYILRVTAYKEGYGEGCVTIHEIENNTDELKFSNCVEEEIYPNNTVTCNDYT
VNQEEYGGAYTSRNRGYNEAPSVPADYASVYEEKSYTDGRRENPCELNRGYRDYTPLPVGYV
TKELEYFPETDKVWIEIGETEGTFIVDSVELLLMEE
(Four different peptide insertions at 282/283, 368/369, 508/509 and
523/524)
SEQ ID NO: 98
MDNNPNINECIPYNCLSNPEVEVLGGERIETGYTPIDISLSLTQFLLSEFVPGAGFVLGLVD
IIWGIFGPSQWDAFLVQIEQLINQRIEEFARNQAISRLEGLSNLYQIYAESFREWEADPTNP
ALREEMRIQFNDMNSALTTAIPLFAVQNYQVPLLSVYVQAANLHLSVLRDVSVFGQRWGFDA
ATIMPLRYPTQVTLDNSRYNDLTRLIGNYTDHAVRWYNTGLERVWGPDSRDWIRYNQFRREL
TLTVLDIVSLFPNYDSRTYPIRTVSQLTREIYTNPVLENFDGSFRGSAQGIEGSIRSPHLMD
ILNSITIYTDAHRGEYYWSGHQIMASPVGFSGPEFTFPLYGTMGNAAPQQRIVAQLGQGVYR
TLSSTLYREPELDNARVLQIRPFNIGINNQQLSVLDGTEFAYGTSSNLPSAVYRKSGTVDSL
DEIPPQNNNVPPRQGFSHRLSHVSMFRSGESNSSVSIIRAPMFSWIHRSAEFNNIIASDSIT
QIPAVKGNFLFNGSVISGPGFTGGDLVRLNSSGNNIMADQTMGLHHGMQNRGYIEVPIHFPS
TAPTTWFNSDSITSTRYRVRVRYASVTPIHLNVNWGNSSIFSNTVPATATSLDNLQSSDEGY
FEGANAFTSSLGDIVGVRNFSGTAGVIIDRFEFIPVTATLEAEYNLERAQKAVNALFTSTNQ
LGLKTNVTDYHIDQVSNLVTYLSDEFCLDEKRELSEKVKHAKRLSDERNLLQDSNEKDINRQ
PERGWGGSTGITIQGGDDVFKENYVTLSGTFDECYPTYLYQKIDESKLKAFTRYQLRGYIED
SQDLEIYSIRYNAKHETVNVPGTGSLWPLSAQSPIGKCGEPNRCAPHLEWNPDLDCSCRDGE
KCAHHSHHFSLDIDVGCTDLNEDLGVWVIFKIKTQDGHARLGNLEFLEEKPLVGEALARVKR
AEKKWRDKREKLEWETNIVYKEAKESVDALFVNSQYDQLQADTNIAMIHAADKRVHSIREAY
LPELSVIPGVNAAIFEELEGRIFTAFSLYDARNVIKNGDENNGLSCWNVKGHVDVEEQNNQR
SVLVVPEWEAEVSQEVRVCPGRGYILRVTAYKEGYGEGCVTIHEIENNTDELKESNCVEEEI
YPNNTVTCNDYTVNQEEYGGAYTSRNRGYNEAPSVPADYASVYEEKSYTDGRRENPCELNRG
YRDYTPLPVGYVTKELEYFPETDKVWIEIGETEGTFIVDSVELLLMEE

The aforementioned exemplary modified Cry1Ac peptide sequences in accordance with the claimed invention are provided for the purpose of representation and should not construed to be limiting.

### Example 2: Recombinant Expression of Native and Modified Cry1Ac Proteins and Partial Purification

### 2.1 Confirmation of Protein Expression

All modified Cry1Ac clones were confirmed for insertion of peptides and the correct open reading frames by sequencing. For primary culture, E. coli cells (DH5-α) harbouring recombinant plasmids (pKK223-3) were inoculated into 6 ml LB medium containing Ampicillin (50 μg/ml) and grown overnight at 37° C. with continuous shaking at 180 rpm. Secondary culture of 50 ml LB (Ampicillin @50 μg/ml) was inoculated with 1% volume of the primary culture and incubated at 37° C./200 rpm. Culture was grown until the OD at 600 nm reached to 0.6. Then, the flasks were shifted to 28° C./200 rpm for 24 h. After 24 h, culture medium was centrifuged at 5000 g for 10 minutes. Pellets were stored at −20° C. until further use.

Out of 50 ml culture medium, 1 ml was processed to check the protein expression. Remaining culture medium was centrifuged at 8,000 g for 10 min. Supernatant was discarded and the pellet was stored at −20° C. till further use. Pellet of 1 ml culture was resuspended in 0.9% NaCl. Centrifugation was carried out again at 8,000 g for 10 minutes and the supernatant discarded. Pellet was resuspended in 1 mL PBS by vortexing. Sample was then sonicated for 5 sec (1 pulse @ 30% amplitude) to lyse the cells. Cell lysate (80 μL) was mixed with 20 μL loading dye and heated at 98° C. for 10 min. Sample was then cooled for 5 min at room temperature and centrifuged at 8,000 g for 10 min. Supernatant (40 μL) was loaded onto SDS (12% resolving) gel. Electrophoresis was carried out at constant current of 20 mA. After electrophoresis, gel was stained with Coomassie Brilliant Blue R-250 and de-stained with methanol and acetic acid. After confirmation of protein expression in pilot culture, pellet obtained from bulk cultures was processed for purification of inclusion bodies (IB).

### 2.2 Purification of Cry1Ac Inclusion Bodies

The frozen cell pellets were thawed and 1 g pellet was resuspended in 50 ml lysis buffer (50 mM Tris-HCL, 50 mM EDTA, 15% Sucrose, pH 8.0) containing Lysozyme (2 mg/ml)). The suspension was mixed well by vortexing and incubated at 37° C. for 1h with constant shaking. The suspension was then homogenized using tissue lyser (@ frequency level-25) for 15 min. This was followed by sonication for 10 min (10 sec pulse @ 30% amplitude). The resultant lysate was centrifuged at 10,000 xg for 15 min at 4° C. The supernatant was discarded, and the pellet vigorously washed with TritonX-100 buffer (2% triton in 0.5 m NaCl). The lysate was centrifuged for 10,000 xg for 15 min at 4° C., washed with TritonX-100 thrice.

The resulting pellet was washed with 25 mL of 0.5 M NaCl solution and mixed well by pipetting. The suspension was vigorously vortexed for 5 min and centrifuged for 15000 xg for 15 min at 4° C. This step (wash with 0.5 M NaCl) was repeated 5 times. All washed fractions (supernatants of each wash) were retained for SDS-PAGE analysis.

Distilled water 25 mL was added to the pellet and mixed by vigorous vortexing for 5 min and then centrifuged for 15000 xg for 15 min at 4° C. This step was repeated once. The pellet obtained after the final wash containing purified IBs were solubilised in 5 mL of solubilisation buffer (50 mM sodium carbonate, pH 8.0) by vigorous mixing or by vortexing for 5 min. The mixture was incubated at 37° C. for 2-3 h with constant shaking at 200 rpm and then centrifuged at 15000 xg for 15 min at room temperature. The supernatant was collected and the solubilized IBs were analysed on SDS-PAGE and stored at −20° C. for future use.

### 2.3 Collection and Artificial Rearing of Cry1Ac Resistant Population of PBW

Pink Bollworm larvae were collected for the purpose of testing and validation from Bt cotton fields from historically known hot spot (Akola in Maharashtra) for PBW attack. The Bt-cotton plants were confirmed by using Cry1Ac lateral flow strips (LFS). Approx. 16,000 mature Bt-cotton balls showing larval burrow signs, pores or feeding larvae were collected and brought back to the insectary (heavily infested plants and balls are shown in FIG. 6 Panel-A). A special tray was designed, and infected balls were arranged to ensure that the larvae will drop in the tray below (as shown in FIG. 6 Panel-D).

The larvae were carefully removed from the tray and transferred on Artificial Diet (AD). First generation was maintained on AD containing 0.1 ppm partially purified Cry1Ac protein. The concentration of Cry1Ac was stepwise increased for each of the future generations. Larvae to pupae progression, mating of adults, collection and hatching of eggs was performed as per the standard practice for PBW artificial rearing. As the concentration of Cry1Ac increased, larval growth and molting was affected. Sometimes, larval duration extended more than normal. Pupation, adult emergence was also severely affected (especially at higher concentrations). However, despite of the conditions, the population of PBW increased slowly. Generation of Cry1Ac resistant PBW population, details of toxin concentration, number of larvae per generation, and larval number obtained is shown in Table 1 below.

TABLE 1

| | No. of | Artificial Diet | Cry1Ac | |
|---|---|---|---|---|
| Generation | Larvae | (AD) composition | Concentration | |
| P1 (Field collected) | ~16000 | BGII ball | — | |
| P2 | ~15000 | AD + Cry1Ac | 0.1 | ppm |
| P3 | ~14000 | AD + Cry1Ac | 1 | ppm |
| P4 | ~13170 | AD + Cry1Ac | 2 | ppm |
| P5 | ~7160 | AD + Cry1Ac | 5 | ppm |
| P6 | ~1000 | AD + Cry1Ac | 10 | ppm |

The larvae once stabilized on 10 ppm of Cry1Ac were constantly maintained on the same concentration for further generations and were used for feeding bioassays as required.

### 2.4 Efficacy of Modified Cry1Ac Proteins Against Resistant PBW Larvae

The modified toxins were incorporated in the artificial diet and feeding bioassays carried out against susceptible and resistant PBW larvae. The native Cry1Ac protein was used as a control. The PBW larvae were fed on artificial diet (AD) and AD incorporated with native Cry1Ac protein (5 ppm) or modified Cry1Ac proteins (5 ppm). The larval growth and mortality were monitored every day.

### 3. Results

### 3.1 Identification of Gut Binding Peptides (GBPs) in Resistant PBW Larvae

Using phage display library, a list of several gut binding peptides was obtained. List of all abundant peptides along with the codon optimized nucleotide sequence is listed in Table 2 below. Out of which four peptides (AIS, APT, SAN and WAM) were randomly selected for the insertion and further studies.

TABLE 2

| SEQ ID | Amino acid | |
|---|---|---|
| NO | sequence | Nucleotide sequence of Peptides |
| 1 | MPLRYPTQVTLD | ATGCCTCTGAGGTATCCGACTCAGGTTACGCTGGAT |
| 2 | DGSMLNRMRGFS | GATGGTTCTATGCTTAATCGGATGCGTGGTTTTAGT |
| 3 | APTTWFNSDSIT | GCGCCTACTACGTGGTTTAATAGTGATTCGATTACT |
| 4 | FKQDAWEAVDIR | TTTAAGCAGGATGCGTGGGAGGCTGTTGATATTCGT |
| 5 | EPELDNARVLQI | GAGCCGGAGTTGGATAATGCTCGGGTGCTTCAGATT |
| 6 | MADQTMGLHHGM | ATGGCGGATCAGACGATGGGGCTGCATCATGGTATG |
| 7 | AFSTTHRYSELI | GCTTTTTCGACGACTCATAGGTATAGTGAGCTGATT |
| 8 | STMTESIRQRWD | TCTACTATGACTGAGAGTATTCGTCAGCGGTGGGAT |
| 9 | AISPSRYFYDET | GCGATTTCGCCGTCGCGTTATTTTTATGATGAGACG |
| 10 | FKQDAWEAVDIR | TTTAAGCAGGATGCGTGGGAGGCTGTTGATATTCGT |
| 11 | EPELDNARVLQI | GAGCCGGAGTTGGATAATGCTCGGGTGCTTCAGATT |
| 12 | YALGRPSLQGPN | TATGCTCTTGGTAGGCCGTCTCTTCAGGGGCCGAAT |
| 13 | DGSMLNRMRGFS | GATGGTTCTATGCTTAATCGGATGCGTGGTTTTAGT |
| 14 | DNHTTDLWNFSH | GATAATCATACGACGGATCTTTGGAATTTTTCTCAT |
| 15 | QQMHEWYFQSLP | CAGCAGATGCATGAGTGGTATTTTCAGAGTTTGCCG |
| 16 | MPLRYPTQVTLD | ATGCCTCTGAGGTATCCGACTCAGGTTACGCTGGAT |
| 17 | STTAVSPVNSAK | TCTACTACTGCTGTGTCTCCTGTGAATTCTGCTAAG |
| 18 | APTTWFNSDSIT | GCGCCTACTACGTGGTTTAATAGTGATTCGATTACT |
| 19 | QELHYGLSSKRF | CAGGAGCTTCATTATGGGCTTTCTAGTAAGCGTTTT |
| 20 | SVSVNNATLYFY | TCGGTTAGTGTTAATAATGCGACTTTGTATTTTTAT |
| 21 | HGDGIKMMRQRM | CATGGTGATGGTATTAAGATGATGCGTCAGAGGATG |
| 22 | SMPRLWDNTSNT | TCTATGCCGCGGCTTTGGGATAATACGTCTAATACG |
| 23 | SLELKRSDVQHA | AGTCTTGAGCTTAAGAGGAGTGATGTGCAGCATGCG |
| 24 | AMTDPWLSDSRS | GCGATGACGGATCCTTGGTTGTCGGATTCTAGGTCG |
| 25 | YSTVYPVGQFPQ | TATTCTACTGTTTATCCTGTTGGTCAGTTTCCTCAG |
| 26 | TTLWLDRDEALK | ACTACTTTGTGGCTTGATAGGGATGAGGCTTTGAAG |
| 27 | FSTDYAWTAEAT | TTTTCTACTGATTATGCGTGGACGGCTGAGGCGACG |
| 28 | SANYNVQAGWTH | TCGGCGAATTATAATGTGCAGGCTGGGTGGACGCAT |
| 29 | SPKSMTTGTVVV | TCTCCTAAGTCGATGACGACTGGGACGGTGGTTGTG |
| 30 | MADQTMGLHHGM | ATGGCGGATCAGACGATGGGGCTGCATCATGGTATG |
| 31 | ASSPRHSFAMGS | GCTAGTAGTCCTCGGCATAGTTTTGCTATGGGGTCT |
| 32 | ISNAVNEIRTLR | ATTAGTAATGCGGTTAATGAGATTCGTACGCTTAGG |
| 33 | AHNEDKWVTHRS | GCTCATAATGAGGATAAGTGGGTTACTCATAGGTCT |
| 34 | GTVSYMVKMDNN | GGTACTGTGTCGTATATGGTGAAGATGGATAATAAT |
| 35 | FNSDSRSTHQED | TTTAATTCTGATTCGCGTAGTACTCATCAGGAGGAT |
| 36 | NQCKECFIRAGD | AATCAGTGTAAGGAGTGTTTTATTCGGGCGGGGGAT |
| 37 | FGLEPRANLHFT | TTTGGTCTGGAGCCGAGGGCGAATCTGCATTTTACT |
| 38 | KIYTITSSHDSY | AAGATTTATACGATTACGTCGAGTCATGATTCTTAT |
| 39 | SNSHPNWVELRP | TCGAATTCTCATCCGAATTGGGTTGAGCTTCGGCCT |
| 40 | WDSLSYPWRITL | TGGGATTCTCTTTCTTATCCTTGGAGGATTACTCTG |
| 41 | IATQKIHVDTKQ | ATTGCTACTCAGAAGATTCATGTTGATACTAAGCAG |
| 42 | WDTSWNFPDQPD | TGGGATACTAGTTGGAATTTTCCTGATCAGCCGGAT |
| 43 | GGWQSLWDKPEH | GGGGGTTGGCAGTCGCTTTGGGATAAGCCGGAGCAT |
| 44 | VAGNKSVSTSHI | GTGGCTGGTAATAAGTCGGTTTCTACTTCGCATATT |
| 45 | GHAHSLTQNPLF | GGGCATGCTCATTCGCTGACGCAGAATCCGTTGTTT |
| 46 | EQWYYSPPIYAN | GAGCAGTGGTATTATTCTCCTCCTATTTATGCTAAT |
| 47 | VDKQHHTAVFPH | GTTGATAAGCAGCATCATACTGCGGTTTTTCCTCAT |
| 48 | VDIKSHFSHTGK | GTGGATATTAAGTCTCATTTTTCGCATACTGGGAAG |
| 49 | VVDPNHSVWILS | GTTGTTGATCCTAATCATAGTGTTTGGATTCTGAGT |
| 50 | FSDAGNLPANAM | TTTTCTGATGCTGGTAATTTGCCGGCTAATGCTATG |
| 51 | HTKLLTYTKSTG | CATACGAAGTTGCTGACGTATACGAAGAGTACTGGT |
| 52 | YPTTANRMYTSY | TATCCTACTACGGCTAATAGGATGTATACTAGTTAT |
| 53 | IGKSSNAPSLRA | ATTGGGAAGTCTTCGAATGCTCCGAGTTTGCGTGCT |
| 54 | DGQVSDHSYVQW | GATGGTCAGGTTAGTGATCATAGTTATGTTCAGTGG |
| 55 | DKYEFFSPTQHY | GATAAGTATGAGTTTTTTTCGCCTACGCAGCATTAT |
| 56 | SLQTLRLGHNTS | TCTCTGCAGACTCTGAGGCTTGGGCATAATACTAGT |
| 57 | WDMWPSMDWKAE | TGGGATATGTGGCCGTCTATGGATTGGAAGGCGGAG |
| 58 | AEPVRDRTQTPK | GCGGAGCCTGTGCGTGATAGGACTCAGACTCCTAAG |
| 59 | EMHKRVLTWNMI | GAGATGCATAAGCGGGTTCTGACTTGGAATATGATT |
| 60 | SVFSSFAIFMDH | TCTGTGTTTTCTAGTTTTGCGATTTTTATGGATCAT |
| 61 | QIEPGDLEHYPT | CAGATTGAGCCTGGGGATTTGGAGCATTATCCTACG |
| 62 | VNRSGIETGAVK | GTTAATCGGAGTGGTATTGAGACGGGGGCTGTGAAG |
| 63 | KVSMQADFWSVM | AAGGTTTCTATGCAGGCTGATTTTTGGAGTGTGATG |
| 64 | EMHGMPLPMLLA | GAGATGCATGGTATGCCTTTGCCTATGCTTCTTGCT |
| 65 | NGAYSLAIRYTS | AATGGTGCTTATAGTCTGGCGATTAGGTATACTTCT |
| 66 | WAMDGQQHSNNY | TGGGCTATGGATGGGCAGCAGCATAGTAATAATTAT |
| 67 | GATSVGSVAMQN | GGGGCTACGAGTGTTGGGAGTGTTGCGATGCAGAAT |
| 68 | AYAAMGGPSQEK | GCTTATGCTGCGATGGGTGGGCCGTCTCAGGAGAAG |
| 69 | SLKPPMHYANMY | TCTCTTAAGCCGCCGATGCATTATGCGAATATGTAT |
| 70 | SYDGTMLKQVRL | AGTTATGATGGTACTATGCTGAAGCAGGTTCGGCTT |
| 71 | LDMYSYIETYNS | CTTGATATGTATAGTTATATTGAGACGTATAATAGT |
| 72 | THDMGKMDRTTT | ACGCATGATATGGGTAAGATGGATCGGACGACTACT |
| 73 | ADNVLRRALENI | GCGGATAATGTGCTTCGGCGTGCGCTTGAGAATATT |
| 74 | SVPAKKAIDWYS | TCGGTTCCGGCTAAGAAGGCGATTGATTGGTATTCG |
| 75 | VHASGFRDSQRW | GTGCATGCGAGTGGTTTTCGGGATTCGCAGAGGTGG |
| 76 | THKLSDWEDIGV | ACACATAAGCTGTCTGATTGGGAGGATATTGGTGTG |
| 77 | GSHNTYIESTIP | GGGTCTCATAATACTTATATTGAGAGTACTATTCCG |
| 78 | GFPVPTKEGNRH | GGGTTTCCTGTGCCGACTAAGGAGGGTAATAGGCAT |
| 79 | IPLENQHKIYST | ATTCCTCTTGAGAATCAGCATAAGATTTATTCGACG |
| 80 | SKISNMQGLHRT | TCGAAGATTTCTAATATGCAGGGTTTGCATCGGACT |
| 81 | KDGMPWNIGYSR | AAGGATGGGATGCCGTGGAATATTGGTTATTCTCGT |
| 82 | QSRTYSGAELLT | CAGTCTAGGACTTATAGTGGGGCTGAGTTGTTGACG |

### 3.2 Identification of Peptide Insertion Sites in Cry1Ac

The coding sequence of Cry1Ac contains 1179 amino acid residues. Based on the data obtained in molecular docking studies, 10 potential amino acid residues (189, 223, 282, 334, 368, 469,508, 523, 666, 834) were identified where insertion of peptides can be done. Table 3 below lists down the residues identified for peptide incorporation based on ZRANK scores wherein residues with ZRANK value <−67.00 were selected (for Native Cry1Ac ZRANK Score was—104.00).

TABLE 3

| | Amino acid | |
|---|---|---|
| S.N. | location | Details regarding modifications |
| 1. | Cry1Ac_189 | Wild type Cry1Ac (peptide insertion between aa |
| | | residue 189 and 190) |
| 2. | Cry1Ac_223 | Wild type Cry1Ac (peptide insertion between aa |
| | | residue 223 and 224) |
| 3. | Cry1Ac_282 | Wild type Cry1Ac (peptide insertion between aa |
| | | residue 282 and 283) |
| 4. | Cry1Ac_334 | Wild type Cry1Ac (peptide insertion between aa |
| | | residue 334 and 335) |
| 5. | Cry1Ac_368 | Wild type Cry1Ac (peptide insertion between aa |
| | | residue 368 and 369) |
| 6. | Cry1Ac_479 | Wild type Cry1Ac (peptide insertion between aa |
| | | residue 479 and 480) |
| 7. | Cry1Ac_508 | Wild type Cry1Ac (peptide insertion between aa |
| | | residue 508 and 509) |
| 8. | Cry1Ac_523 | Wild type Cry1Ac (peptide insertion between aa |
| | | residue 523 and 524) |
| 9. | Cry1Ac_666 | Wild type Cry1Ac (peptide insertion between aa |
| | | residue 666 and 667) |
| 10. | Cry1Ac_834 | Wild type Cry1Ac (peptide insertion between aa |
| | | residue 834 and 835) |

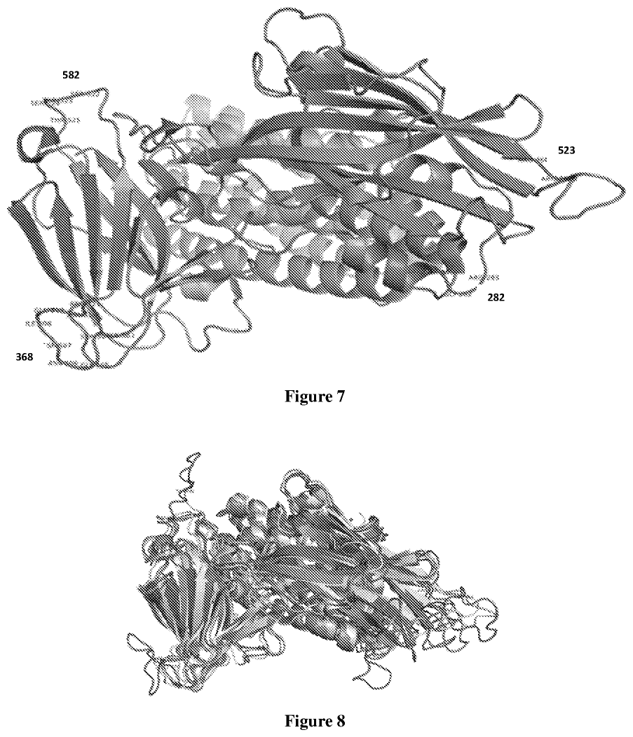

Among the identified sites, four sites viz. 282, 368, 523 and 582 showed most stable protein conformation after insertion of peptide in in-silico studies. Some representative insertion sites (amino acid residue) with inserted peptide(s) viz. 282, 368, 523 and 582 were used during in silico studies are shown in FIG. 7 and the resulting sequence post-insertion of peptides as shown in Table 4.

TABLE 4

| | Peptide (in bold) | Insertion |
|---|---|---|
| S.N. | inserted region | Site |
| 1 | DGSFRGAISPSRYFY | 282 |
| | DETSAQGI | |
| 2 | NSSGNNIAPTTWFNS | 368 |
| | DSITQNRGYI | |
| 3 | SSTLYRSANYNVQAG | 523 |
| | WTHRPFNIGI | |
| 4 | PIHFPSTWAMDGQQH | 582 |
| | SNNYSTRYRVRV | |

The four selected peptides were inserted at the sites identified in Cry1Ac either one at a time and or multiple and several toxin models were constructed. These models were further docked with wild type and mutant cadherin models and checked for energy.

Overlaying of the predicted structures suggested that the core Cry1Ac structure is not affected by the incorporation of peptide(s) in the loops. FIG. 8 shows overlay of modified toxins with insertion of peptide at different positions.

From these studies, it was concluded that potential sites exist in the coding sequence of Cry1Ac wherein gut binding peptides of 12 amino acid residues (36 nucleotides) may be introduced without altering the structure and conformation of the toxin.

### 3.3 Modifications of Cry1Ac by Insertion of Single Peptide

Various different modifications were achieved by insertion of GBPs at specific sites in Cry1Ac using overlap extension PCR method (Bryksin and Matsumara, 2018). The steps involved and results obtained in PCR, agarose gel electrophoresis, high resolution melt analysis are shown in FIG. 9. That is by insertion of typical peptide (SANYNVQAGWTH) as following in FIG. 9:
Representative (a) shows generation of two different overlapping fragments
Representative (b) shows joining of two fragments using overlap extension PCR
Representative (c) shows validation of mutation using mutation specific PCR
Representative (d) shows HRM profile of mutated fragment.

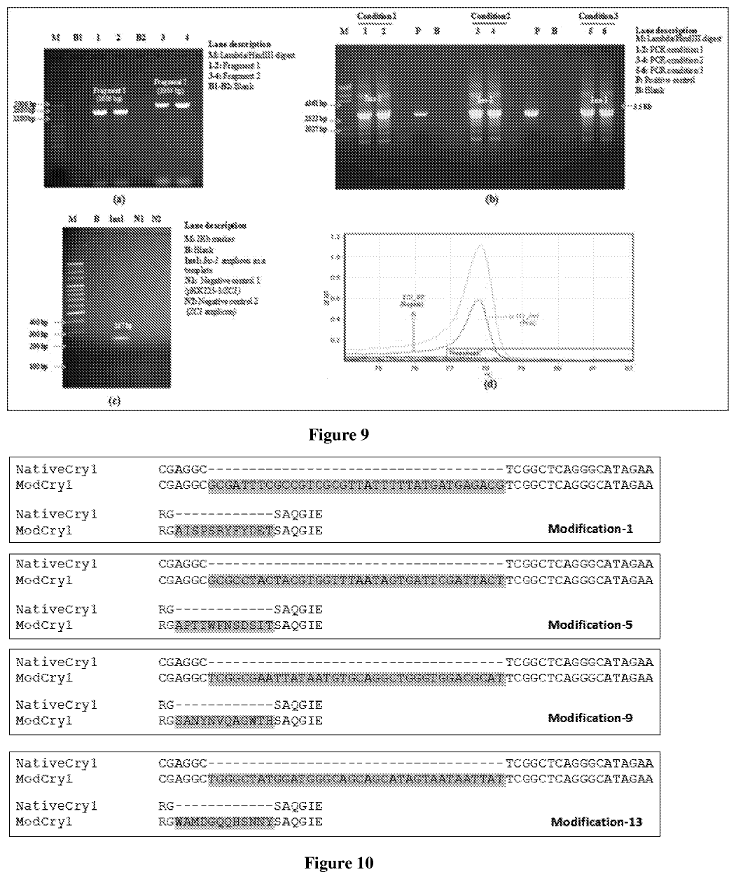

The successful insertions of peptides in the Cry1Ac were confirmed by sequencing the amplified PCR products. The DNA sequences of modified Cry1Ac toxins showed insertion of 36 nucleotides at the designated insertion site. As a representation, sequence alignments of native and modified toxin for the region of insertion for modifications (AIS @ 282, APT @ 282, SAN @ 282 and WAM @ 523) are shown in FIG. 10.

### 3.4 Modifications of Cry1Ac by Insertion of Multiple Peptides

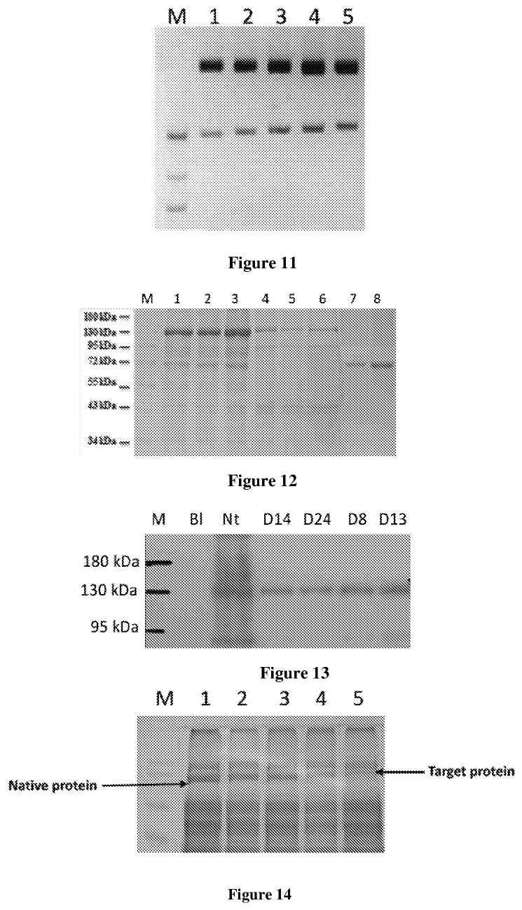

The modified Cry1Ac(s) with multiple peptide insertions (No. 17 to No. 22) were also generated using OLE PCR technique. The successful insertions were confirmed initially by agarose gel electrophoresis or by gel shift assays (FIG. 11). The restriction digestion and agarose gel electrophoresis analysis showed expected increment in the molecular weight of DNA.

### 3.5 Recombinant Expression of Native and Modified Cry1Ac Proteins and Partial Purification

All Cry1Ac(s) modified by incorporation of peptide were cloned in vector pKK223-3 and expressed in E. coli. Partially purified native and modified Cry1Ac proteins were analysed by SDS-PAGE followed by Coomassie staining. The results are shown in FIG. 12. Predicted molecular weight of Cry1Ac is approx. 133.12 kDa. For native Cry1Ac a band of predicted molecular weight was visible. Results for one modified recombinant Cry1Ac with single peptide insertion are shown here. The predicted molecular weight of modified (peptide inserted) Cry1Ac is approx. 134.66 kDa (addition of peptide increases the molecular weight by approximately 1.5 kDa). This can also be visually confirmed by the shift in mobility of the modified Cry1Ac. The partially purified proteins from IBs were visually quantified using known concentrations of BSA as standards and used for insect feeding bioassays.

The total proteins from bacterial lysate were separated by SDS-PAGE on 12% acrylamide gels; sample preparation, running of gels, staining and transfer to nitrocellulose membranes were performed as described earlier (Fitches et al., 2004). Primary (anti Cry1Ac) antibodies used in Western blotting were diluted to 1:5000 for incubation with blots. The detection of immunoreactive bands was done using chemiluminescent detection kit. The western blot results showed immunoreactive bands at the predicted molecular weight for native (133.12 kDa) and modified Cry1Ac (134.65 kDa).

3.6 Recombinant Expression of Cry1Ac with Multiple Peptide Insertions

The Cry1Ac(s) modified with multiple peptide insertions (No. 17 to No. 22) showed successful recombinant expression as well as expected migration shift in the SDS-PAGE analysis (FIG. 14).

### 3.7 Efficacy of Modified Cry1Ac Proteins Against Cry1Ac Resistant PBW Larvae

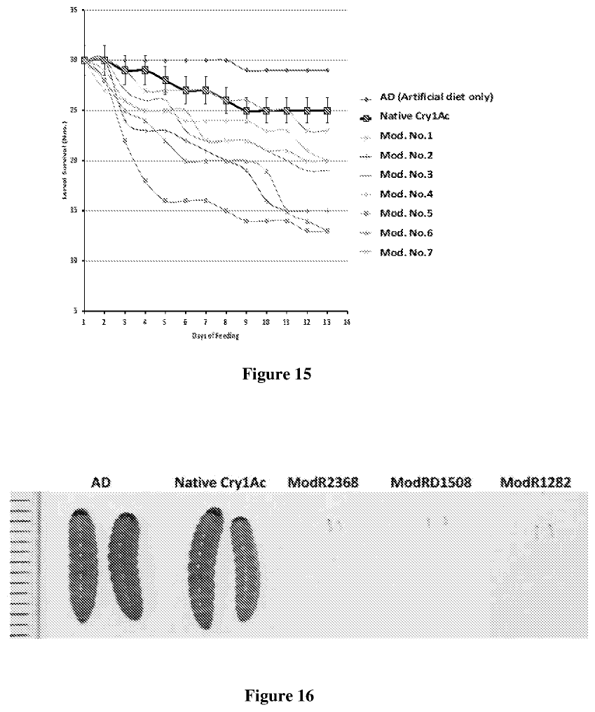

The susceptible larvae feeding on native and modified Cry1Ac proteins showed 100% mortality. The results of insect feeding bioassays are shown in FIG. 15.

All modified toxins (No. 1 to No. 7) showed increased toxicity to resistant PBW than the native Cry1Ac. By day 10 of the bioassay, larvae fed on modified Cry1Ac Mod. No. 5 showed about 50% mortality, whereas 13% larval mortality was recorded on native Cry1Ac. The results showed that peptide insertion did not affect protein toxicity rather it significantly increased protein toxicity against Cry1Ac resistant PBW larvae.

Moreover, survived larvae showed significant growth retardation as shown in FIG. 16. Larvae were carefully removed from the diet cubes on 13^{th} day of the assay and growth data was recorded.

The Cry1Ac(s) inserted with multiple peptides showed similar levels of toxicity as single insertion Cry1Ac(s) against resistant PBW. The combinations of different gut binding peptides and their insertion at different sites in Cry1Ac may be used further to enhance the toxicity of Cry1Ac. Thus, the present invention has tremendous potential to address the problem of PBW resistance to native Cry1Ac. The modified toxins when expressed in cotton plants could substantially tackle the resistant population of multiple geographies.

### Example 4: Generation of Transgenics

### 4.1 Design and Construction of Binary Vector Expressing Cry1Ac and its Modified Versions

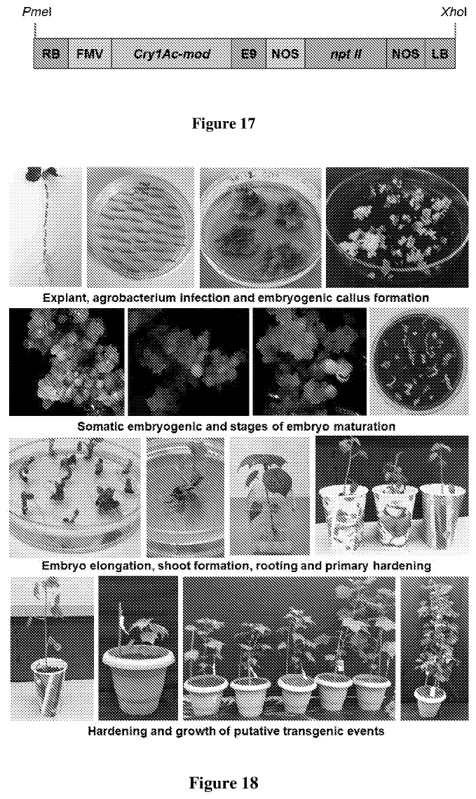

The Cry1Ac gene sequence from B. thuringiensis and its modified versions were codon optimized using codon usage for cotton. A construct containing codon optimized gene sequences coding for Cry1Ac, and its modified versions were synthesized from GenScript, USA. The Cry1Ac gene was expressed under figwort mosaic virus (FMV) 35 s promoter and a selectable marker neomycin phosphotransferase II (nptII) gene was expressed under Nopaline synthase (NOS) promoter (FIG. 17). A complete synthesized construct sequence was cloned in an empty vector using Pmel-Xhol restriction sites. The presence of inserted gene and stability of the final constructs was verified by restriction digestion and sequencing. The resultant binary vector was named pASPL818 and mobilized into Agrobacterium strain LBA4404.

### 4.2 Agrobacterium-Mediated Transformation and Plant Regeneration

Agrobacterium tumefaciens strain LBA4404 was utilized for transformation. The strain harboring a binary vector, pASPL818 carrying Cry1Ac gene sequences was cultured overnight at 28° C. in Luria-Bertani (LB) medium supplemented with 50 mg/L kanamycin, 50 mg/L rifampicin, and 250 mg/L streptomycin. The bacterial cells were then collected by centrifugation, washed twice with 10 mM magnesium chloride, and resuspended in liquid MS medium supplemented with 200 μM acetosyringone and achieved a final optical density (OD600) of 0.8. Seeds of cotton cv. Coker 312 were surface sterilized by 70% ethanol treatment for 1 minute, followed by immersion in 1% sodium hypochlorite for 10 minutes, and washed with sterile distilled water. The seeds were germinated in culture bottles containing MS medium supplemented with 30 g/L sucrose and 8 g/L agar. Hypocotyls from 8-9 days old seedlings were used as explants. The explants were co-cultivated with the Agrobacterium suspension for 72 hours under a photoperiod of 16 hours of light and 8 hours of dark at 25° C. on MS medium supplemented with 100 μM acetosyringone, 0.5 mg/L 2,4-Dichlorophenoxyacetic acid (2,4-D) and 0.1 mg/L Zeatin. Followed by co-cultivation, the explants were washed with Cefotaxime (300 mg/L) and placed on selection medium consisting of MS medium supplemented with 30 g/L sucrose, 8 g/L agar, 0.5 mg/L 2,4-D, 0.1 mg/L Zeatin, 50 mg/L kanamycin and 400 mg/L carbenicillin. The explants were then incubated at 28° C. with a 16-hour photoperiod for 4 weeks, with the selection medium replaced every 2 weeks. The process was continued for 3-4 cycles (4 weeks each) of sub-culturing. Kanamycin-resistant calli were observed and shifted to embryo induction medium containing MS medium supplemented with 30 g/L sucrose, 8 g/L agar, 0.1 mg/L 2,4-D, 0.1 mg/L Zeatin and 50 mg/L kanamycin. The cultures were further incubated at 28° C. with a 16-hour photoperiod for 4 weeks. Embryogenic calli were then selectively placed on embryo proliferation medium containing MS medium supplemented with 30 g/L sucrose, 8 g/L agar, 0.1 mg/L Zeatin, 0.1 mg/L indol acetic acid (IAA) and 50 mg/L kanamycin and incubated at 28° C. until different embryonic developmental stages observed. Cotyledonary embryos were harvested and placed on embryo elongation medium containing half strength MS medium supplemented with 30 g/L sucrose, 8 g/L agar and 50 mg/L kanamycin. Elongated embryos with completely developed shoots and roots were taken for primary hardening. Primary hardened plants were then transferred to soil in pots and acclimatized in a growth chamber at 28° C. with a 16-hour photoperiod for further growth and development.

### 4.3 Pink Bollworm Feeding Bioassays

Leaf disk bioassays were carried out using a Bt-resistant pink bollworm population. The leaf disks were obtained from transgenic cotton plants engineered to express modified Cry1Ac toxins. Each small leaf piece (2 cm²) was put in a separate container for the insects to feed on, with one first-instar pink bollworm larva on each piece. Visual observation of leaf disc consumption by the larvae was carried out regularly, and the mortality of the larvae was documented on a daily basis.

### Example 5: Results and Analysis of Assessment Outcomes

### 5.1 Transformation of Modified CryAc Gene in Cotton

To assess the effectiveness of the modified Cry1Ac, a binary vector carrying the modified gene was constructed and introduced into cotton plants through Agrobacterium-mediated transformation (FIG. 17). A highly friable callus was derived from the hypocotyl of Coker 312,which was subsequently screened for resistance to kanamycin. After 3-4 cycles (4-week) of subculture, somatic embryogenesis was observed. Within 3-5 weeks on the selection medium, globular clusters of embryos formed from 2-3 g of embryogenic calli that had undergone transformation. Each cluster represented an independent transformation event. These embryos progressed to the cotyledonary stage and germinated normally within a few weeks. The majority of the shoots rooted effectively without requiring additional treatment. Upon transfer to greenhouse soil conditions, all plants exhibited regular morphology and underwent flowering (FIG. 18). The gene insertion was confirmed through PCR, while the copy number was determined using southern blot analysis.

### 5.2 Bioassay Analysis

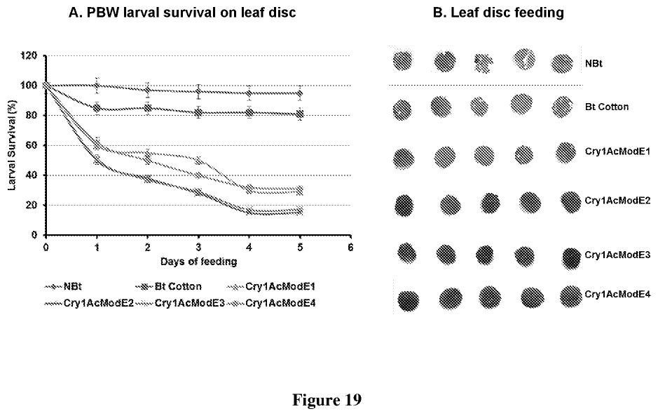

Leaf disc feeding assays were conducted across four distinct transgenic events to assess the enhanced effectiveness of modified toxins in comparison to native Cry1Ac expressed in conventional Bt cotton. The transgenic cotton events, which expressed the modified Cry1Ac toxin, exhibited enhanced toxicity towards resistant pink bollworms. By the third day of the bioassay, larvae across all events displayed approximately 50% mortality, while 18% larval mortality was observed on native Cry1Ac-expressing cotton (FIG. 19A). These findings indicate that peptide insertion did not compromise protein toxicity; in fact, it substantially amplified protein toxicity against Cry1Ac-resistant PBW larvae. On the fifth day, Cry1AcModE2 and Cry1AcModE3 events demonstrated 85% larval mortality, whereas Cry1AcModE1 and Cry1AcModE4 recorded 71% and 69% mortality respectively (FIG. 19A). Concurrently, the mortality rates of larvae on non-Bt cotton (NBt) and conventional Bt cotton were 5% and 19% respectively. Visual observations of leaf disc feeding highlighted a significantly reduced consumption in the transgenic events compared to NBt and Bt cotton (FIG. 19B). Overall, these results emphasize the efficacy of modified toxins expressed transgenic cotton events, providing a valuable tool for combatting Bt-resistant pink bollworms. Consequently, the present invention holds substantial potential in addressing the challenge of PBW resistance to Bt cotton.

### Advantages

1. A unique approach for mitigating/addressing the problem of Bt resistant lepidopteran pests specifically pink bollworm.

2. The invention can be applied to all cotton cultivating geographies facing the above problem.

3. The invention has a high potency to delay field evolved insect resistance and to sustain the Bt cotton technology for a longer duration.

4. The invention has an improved toxicity against pests especially resistant pink bollworm.

1. A modified Cry1Ac protein comprising one or more gut binding peptide (GBP) sequences selected from SEQ ID NO: 1 to 82, inserted at at least one of the amino acid positions selected from 189, 223, 282, 334, 368, 469, 508, 523, 666 and 834 of Cry1Ac, wherein said mutant Cry1Ac protein has improved insecticidal activity/toxicity against Lepidopteran insects specifically Bt resistant pink bollworm (PBW) compared to native Cry1Ac protein.

2. The modified Cry1Ac protein as claimed in claim 1, wherein the insect is pest, pest population or resistant pest species of Lepidopteran insects specifically pink bollworm (PBW).

3. A method of identifying one or more stable target site(s) in native Cry1Ac protein for insertion of a gut binding peptide as defined in claim 1 comprising:
i. in silico studies comprising structural analysis and insertion of known gut binding peptides to check stability of the protein pre and post insertion; and
ii. identification and selection of stable sites based on the stability results.

4. The method as claimed in claim 3, wherein said stable target sites are selected from amino acid positions 189, 223, 282, 334, 368, 469, 508, 523, 666 and 834 of Cry1Ac.

5. A method of preparing the modified Cry1Ac protein as claimed in claim 1, comprising:
a. identifying the gut binding peptides (GBPs) as defined in claim 1;
b. in silico identification of one or more stable target site(s) in Cry1Ac protein for insertion of one or more said gut binding peptide(s); and
c. modification of native Cry1Ac gene by insertion of one or more GBPs at at least one or more identified sites to obtain the modified Cry1Ac protein,
wherein said modified Cry1Ac protein has improved insecticidal activity/toxicity against Lepidopteran insects specifically Bt resistant pink bollworm (PBW) compared to native Cry1Ac protein.

6. The method as claimed in claim 5, comprising:
i. screening a phage display library using biopanning protocol;
ii. feeding the insect larvae on the phage display library using either artificial diet or coated cotton balls;
iii. dissecting of larval guts;
iv. elution for bound phages;
v. amplification and enrichment of eluted phages via transfection into host cells for creating a library of peptides;
vi. analysis of individual clones and sequencing; and
vii. shortlisting gut binding peptides that bind to the gut of the insect, wherein the gut binding peptides frequently appearing in the selected phage population and exhibiting higher phage recovery than other clones were selected.

7. A polynucleotide comprising a nucleic acid molecule encoding the modified Cry1Ac protein(s) as claimed in claim 1.

8. A vector comprising the polynucleotide as claimed in claim 7.

9. A host cell transformed using the vector as claimed in claim 8, wherein said cell is capable of expressing modified Cry1Ac(s) comprising one or more gut binding peptide (GBP) sequences selected from SEQ ID NO: 1 to 82, inserted at at least one of the amino acid positions selected from 189, 223, 282, 334, 368, 469, 508, 523, 666 and 834 of Cry1Ac, wherein said mutant Cry1Ac protein has improved insecticidal activity/toxicity against Lepidopteran insects specifically Bt resistant pink bollworm (PBW) compared to native Cry1Ac protein.

10. The host cell as claimed in claim 9, wherein said host cell is a plant cell.

11. A method of producing an insect-resistant transgenic plant, comprising:
a. stably integrating the polynucleotide as claimed in claim 7 into the genome of a host/plant cell; and
b. obtaining a stably transformed plant from plant/plant cell capable of expressing the modified Cry1Ac, wherein said stably transformed plants are resistant to Lepidopteran insects.

12. A library of gut binding peptides (GBPs) comprising peptides having 12 amino acid residues and defined by SEQ ID NO: 1 to 82 wherein said peptides are capable of binding to one or more gut receptors of Lepidopteran insects.

13. An insecticidal formulation comprising an effective amount of the modified Cry1Ac proteins as claimed in claim 1.

14. A composition comprising the insecticidal formulation as claimed in claim 13 along with one or more formulating agents selected from but not limited to the group consisting of adjuvants, surfactants, thickeners, stickers, petroleum oils, crop oil concentrate, stabilizing agents, solvents, hygroscopic agents, deposit builders, antifoam agents, buffering agents and activators.

15. A method of inhibiting the growth of an insect pest, insect pest population, or resistant lepidopteran insect pests, comprising contacting said insect pest(s) with the modified Cry1Ac protein as claimed in claim 1.

16. A method for improving the yield of a crop using the insecticidal formulation as claimed in claim 13.

17. A transgenic plant cell, plant or plant part carrying the modified Cry1Ac protein as claimed in claim 1.

18. A method for improving the yield of a crop using the composition as claimed in claim 14.
